(19) United States
(12) Patent Application Publication
(10) Pub. No.: US 2025/0186194 A1
(43) Pub. Date: Jun. 12, 2025

(54) PROSTHETIC VALVE

(71) Applicant: EDWARDS LIFESCIENCES CORPORATION, Irvine, CA (US)

(72) Inventors: Michael Bukin, Pardes Hanna (IL); Nikolai Gurovich, Hadera (IL); Elena Sherman, Pardes Hana (IL); Tyler Dale O'Dell, Irvine, CA (US)

(73) Assignee: EDWARDS LIFESCIENCES CORPORATION, Irvine, CA (US)

(21) Appl. No.: 19/056,662

(22) Filed: Feb. 18, 2025

Related U.S. Application Data
(63) Continuation of application No. PCT/US2023/030807, filed on Aug. 22, 2023.
(60) Provisional application No. 63/400,229, filed on Aug. 23, 2022.

(51) Int. Cl. A61F 2/24

(52) U.S. Cl. CPC A61F 2/2415; A61F 2/2418; A61F 2220/0075

(57) ABSTRACT

A prosthetic valve is disclosed comprising an annular frame, a plurality of leaflets positioned within and coupled to the annular frame, each leaflet comprising a body portion having a free outflow edge and two opposing commissure portions on opposite sides of the leaflet, wherein each commissure portion is folded along a radially-extending fold line to form an inner fold layer and an outer fold layer, wherein each commissure portion is paired with a commissure portion of an adjacent leaflet to form a commissure that is coupled to the frame, and for each commissure, a retention member extending between the inner fold layer and the outer fold layer of a first commissure portion of the commissure and between the inner fold layer and the outer fold layer of a second commissure portion of the commissure to hold the first and second commissure portions together during working cycles of the prosthetic valve.

### CROSS-REFERENCE TO RELATED APPLICATION

This application is a continuation of PCT Application No. PCT/US2023/030807, filed on Aug. 22, 2023, which claims the benefit of U.S. Provisional Application No. 63/400,229, filed Aug. 23, 2022, both of which are incorporated by reference herein.

### FIELD

The present disclosure relates to valvular structures for prosthetic valves and to delivery apparatus and methods for implanting prosthetic valves.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (for example, stents) and artificial valves, as well as a number of known methods of implanting these devices and valves in humans. Percutaneous and minimally invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery apparatus and advanced through the patient's vasculature (for example, through a femoral artery and the aorta) until the prosthetic heart valve reaches the implantation site in the heart. The prosthetic heart valve is then expanded to its functional size, for example, by inflating a balloon on which the prosthetic valve is mounted, actuating a mechanical actuator that applies an expansion force to the prosthetic heart valve, or by deploying the prosthetic heart valve from a sheath of the delivery apparatus so that the prosthetic heart valve can self-expand to its functional size.

### SUMMARY

Described herein are prosthetic heart valves, delivery apparatuses, and methods for implanting prosthetic heart valves. The disclosed prosthetic heart valves and methods can, for example, improve the durability and/or longevity of valvular structures within a prosthetic heart valve. The disclosed prosthetic heart valves, for example, can include a retention member at each commissure to hold commissure portions of two adjacent leaflets together, which can prevent the leaflets from contacting the frame and/or prevent leaflet fluttering for prosthetic heart valves operating within a range of working diameters. As such, the devices and methods disclosed herein can, among other things, overcome one or more of the deficiencies of typical expandable prosthetic valves.

In one representative example, a prosthetic valve comprises an annular frame, a plurality of leaflets positions within the coupled to the annular frame.

In some aspects, each of the plurality of leaflets comprises a body portion having a free outflow edge and two opposing commissure portions on opposite sides of the leaflet.

In some aspects, each commissure portion is folded along a radially-extending fold line to form an inner fold layer and an outer fold layer.

In some aspects, each commissure portion is paired with a commissure portion of an adjacent leaflet to form a commissure that is coupled to the frame.

In some aspects, for each commissure, a retention member extends between the inner fold layer and the outer fold of a first commissure portion of the commissure and between the inner fold layer and the outer fold layer of a second commissure portion of the commissure to hold the first and second commissure portions together during working cycles of the prosthetic valve.

In some aspects, the retention member comprises a loop.

In some aspects, the retention member comprises a suture.

In some aspects, the retention member does not pierce any portions of the leaflets.

In some aspects, the retention member extends continuously in a closed loop through the inner and outer fold layers of the first commissure portion, around radial inner edges of the inner fold layers of the first and second commissure portions, through the inner and outer fold layers of the second commissure portion and around radial outer edges of the inner fold layers of the first and second commissure portions.

In some aspects, the retention member extends radially inwardly from the annular frame.

In one representative example, a prosthetic valve comprises an annular frame; a plurality of leaflets positioned within and coupled to the annular frame, each leaflet comprising a body portion having a free outflow edge and two opposing commissure portions on opposite sides of the leaflet, wherein each commissure portion is folded along a radially-extending fold line to form an inner fold layer and an outer fold layer, wherein each commissure portion is paired with a commissure portion of an adjacent leaflet to form a commissure that is coupled to the frame; and for each commissure, a retention member extending between the inner fold layer and the outer fold layer of a first commissure portion of the commissure and between the inner fold layer and the outer fold layer of a second commissure portion of the commissure to hold the first and second commissure portions together during working cycles of the prosthetic valve.

In another representative example, a prosthetic valve comprises an annular frame; and a valvular structure mounted within the annular frame, the valvular structure comprising a plurality of leaflets having commissure portions that define a plurality of commissures and a plurality of retention members, wherein each retention member extends through two radially-extending channels defined by commissure portions of two adjacent leaflets and is configured to hold the commissure portions of the two adjacent leaflets together during working cycles of the valvular structure.

In another representative example, a prosthetic valve comprises an annular frame; a plurality of leaflets positioned within and coupled to the annular frame, each leaflet comprising a free outflow edge and two opposing commissure portions on opposite sides of the leaflet, wherein each commissure portion is paired with a commissure portion of an adjacent leaflet to form a commissure that is coupled to the frame; and for each commissure, a retention member coupled to two adjacent commissure portions to hold the adjacent commissure portions together during working cycles of the prosthetic valve, wherein the retention member extends in a radial inward direction relative to the annular frame.

In another representative example, a delivery apparatus comprises a delivery device comprising a handle; and a prosthetic valve releasably coupled to the delivery device, the prosthetic valve comprising an annular frame and a valvular structure mounted within the annular frame, the valvular structure comprising a plurality of leaflets having fold portions that define a plurality of commissures and a plurality of retention members, wherein each retention member extends through two radially-extending channels defined by fold portions of two adjacent leaflets and is configured to hold the fold portions of the two adjacent leaflets together during working cycles of the valvular structure.

In another representative example, a method of assembling a prosthetic valve, the method comprises forming a valvular structure that includes a plurality of leaflets that define a plurality of commissures and a radially-extending retention member at each commissure; and coupling the valvular structure to a frame.

In another representative example, a prosthetic valve comprises an annular frame; a plurality of leaflets positioned within and coupled to the annular frame, each leaflet comprising a free outflow edge, two opposing primary tabs extending from opposite sides of the leaflet and two opposing secondary tabs disposed at opposite sides of the free outflow edge and extending axially beyond the free edge in downstream direction, wherein each primary tab is paired with a primary of an adjacent leaflet to form a commissure that is coupled to the frame; and at each commissure, a retention member connecting a pair of adjacent secondary tabs of adjacent leaflets to hold the secondary tabs together during working cycles of the prosthetic valve.

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

### BRIEF DESCRIPTION OF THE DRAWINGS

FIG. 1 is a perspective view of a partially assembled valvular structure including leaflets, according to one example.

FIG. 2 is a perspective view of the valvular structure of FIG. 1 with the upper tabs of the leaflets folded down.

FIG. 3A is a flattened view of one of the leaflets of the valvular structure of FIGS. 1-2.

FIG. 3B is a flattened view of the leaflet of FIG. 3A with upper tabs of the leaflet folded down against the lower tabs of the leaflet.

FIG. 4A is a perspective view of a commissure formed from two of the leaflets shown in FIG. 3A and a retaining member.

FIG. 4B is a perspective view of the commissure of FIG. 4A with portions of the commissure shown in cross-section for illustrative purposes.

FIG. 5 is a perspective view of the commissure of FIG. 4A attached to a frame, according to one example.

FIG. 6A is a perspective view of one example of a prosthetic valve that incorporates leaflets of the type shown in FIGS. 1-4 and retaining members of the type shown in FIGS. 4A-4B.

FIG. 6B is a perspective view of the prosthetic valve of FIG. 6A with an outer skirt as a sealing member.

FIG. 7A is a perspective view of a frame of the prosthetic valve of FIGS. 6A-6B.

FIG. 7B is a front portion of the frame shown in FIG. 7A.

FIGS. 8A-8C are schematic illustrations of a process of forming the commissures of a valvular structure.

FIG. 9 is a side elevation view of a delivery apparatus for a prosthetic device, such as a prosthetic valve, according to one example.

FIG. 10 is a perspective view of a portion of an actuator of the prosthetic valve of FIGS. 6-7 and an actuator assembly of a delivery apparatus, according to one example.

FIG. 11 is a perspective view of the actuator and actuator assembly of FIG. 10 with the actuator assembly physically coupled to the actuator.

FIG. 12A is a top view of a prosthetic valve that does not include a retention member, according to one example.

FIG. 12B is a top view of a prosthetic valve that includes a retention member, according to one example.

FIG. 13 is a flattened view of a leaflet of a valvular structure, according to another example.

FIG. 14 is a top view of a commissure formed from two leaflets of the type shown in FIG. 13 and a retaining member, according to one example.

FIG. 15 is a top view of a commissure formed from two leaflets of the type shown in FIG. 13 and a retaining member, according to another example.

FIG. 16 is a partial side view of the commissure shown in FIG. 14.

FIG. 17 is a partial side view of a commissure similar to FIG. 16, wherein the leaflets have an alternate tab portion and a retaining member, according to another example.

### DETAILED DESCRIPTION

### General Considerations

For purposes of this description, certain aspects, advantages, and novel features of examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (for example, out of the patient's body), while distal motion of the device is motion of the device away from the user and toward the implantation site (for example, into the patient's body). The terms “longitudinal” and “axial” refer to an axis extending in the proximal and distal directions, unless otherwise expressly defined.

### Overview of the Disclosed Technology

As introduced above, prosthetic heart valves can include leaflets that are secured to one another at their adjacent sides (for example, commissure tabs) to form commissures which are secured to a frame of the prosthetic heart valve. During the diastolic and systolic phases of the heart, the leaflets cycle between an open state, where leaflet free edges are separated from each other, and a closed state, where the leaflet free edges coapt. In some examples, in the open state, the leaflet free edges can flutter back and forth which may result in contact occurring between the leaflets and the frame. This can place excessive stresses on the leaflets and may cause damage to the leaflets, particularly for prosthetic heart valves operating within a range of working diameters. For example, when the prosthetic heart valve is in the open state, the commissures can undesirably splay apart such that the leaflet free edges are overly slackened, resulting in leaflet fluttering and/or contact between the leaflets and the frame which may decrease the durability of the leaflets.

Described herein are examples of prosthetic implants, such as prosthetic heart valves, that can be implanted within any of the native valves of the heart (for example, the aortic, mitral, tricuspid, and pulmonary valves). The prosthetic valves described herein can have a plurality of leaflets mounted within the frame in such a way that an opening axis of the leaflets is radially inward of the frame and the leaflet free edges are retained away from the frame while still permitted to coapt. For example, the prosthetic heart valves of the present disclosure can include a retention ring that extends in a generally radial or horizontal direction around leaflets at each commissure to securely hold the leaflets together. Advantageously, the retention rings can prevent undesirable fluttering of the leaflets and/or unwanted contact of the leaflets with the frame, thereby improving the durability and/or longevity of the leaflets during operating cycles of the prosthetic heart valve.

The frames described herein can further comprise actuators (for example, expansion mechanisms) and/or locking mechanisms to enable greater control over the radial compression or expansion of the valve body. Axial posts of the disclosed frames can also be configured to move axially toward and contact one another as the frame is radially expanded to limit or prevent over expansion of such valves. The frames can also comprise a plurality of commissure support members to which leaflet tabs or commissures can be radially or axially inserted and attached.

The prosthetic valves disclosed herein can be radially compressible and expandable between a radially compressed configuration and a radially expanded configuration. Thus, the prosthetic valves can be crimped and retained by an implant delivery apparatus in the radially compressed state during delivery and expanded to the radially expanded state once the prosthetic valve reaches the site of implantation. It is understood that the valves disclosed herein may be used with a variety of implant delivery apparatuses, examples of which are discussed in more detail in the following disclosure.

### Examples of the Disclosed Technology

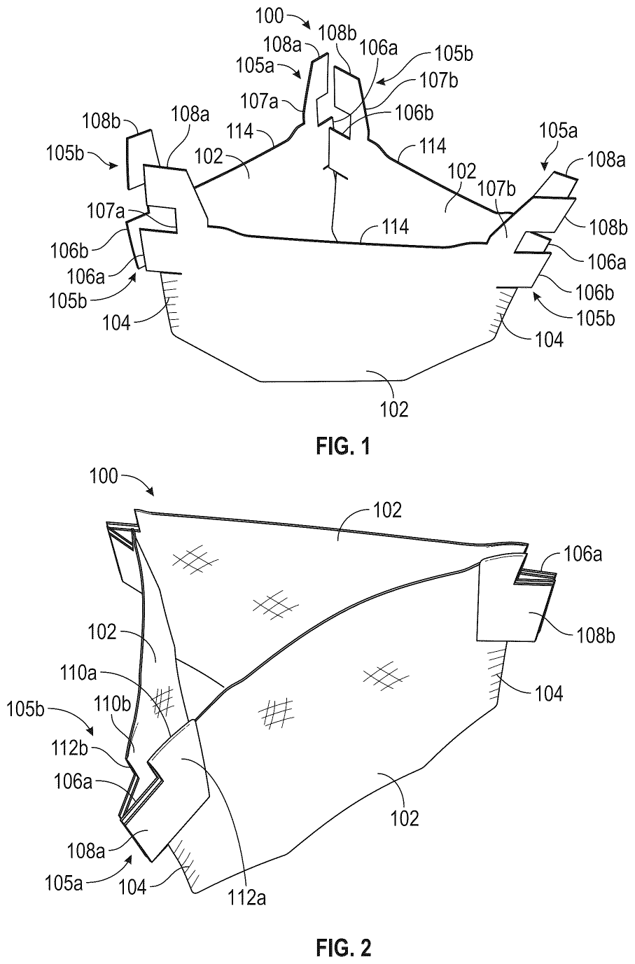

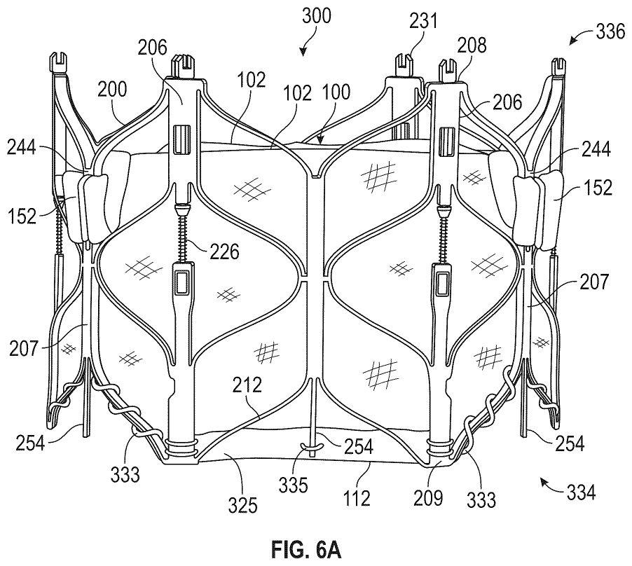

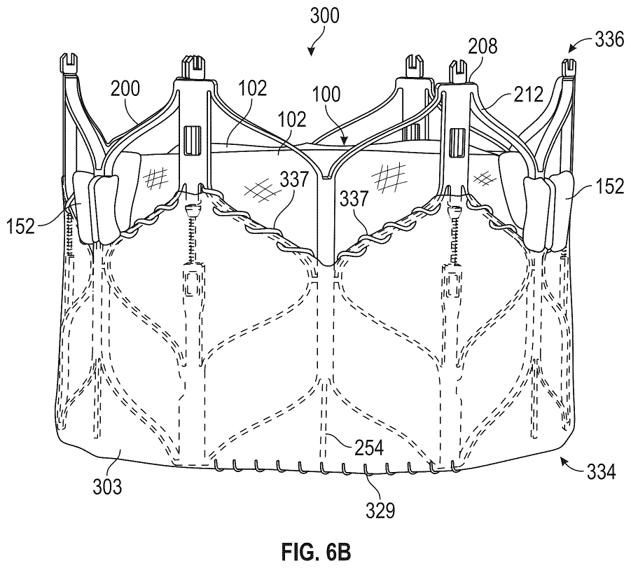

Turning now to the drawings, FIG. 1 shows an exemplary valvular structure 100 (also referred to as a leaflet assembly), in a partially assembled state. The valvular structure 100 may be mounted within an annular frame of a prosthetic valve (for example, valve 300 of FIG. 6A, discussed below). In one illustrative example, the valvular structure 100 includes a plurality of leaflets 102 that in use are coupled to the frame and open and close to regulate flow of blood through the frame. The leaflets 102 have leaflet free edges 114 that move radially to coapt with each other during flow cycles of the heart. The valvular structure 100 is shown as having three leaflets 102 that can be arranged to collapse in a tricuspid arrangement. However, in other examples, the valvular structure 100 may have greater or fewer number of leaflets than three (for example, one or more leaflets). The leaflets 102 are made of a flexible material. In a particular example, the leaflets 102 may be made in whole or in part from pericardial tissue (for example, bovine pericardial tissue), biocompatible synthetic materials, or various other suitable natural or synthetic materials as known in the art and described in, for example, U.S. Pat. No. 6,730,118, the relevant disclosure of which is incorporated herein by reference.

In the partially assembled state shown in FIG. 1, the leaflets 102 may be partially secured together at a portion of their adjacent sides by sutures 104. Suturing of adjacent sides as shown at 104 may occur before or after positioning the leaflets within a frame. Each leaflet 102 includes commissure portions 105a, 105b at opposite sides of the leaflet. The commissure portions 105a, 105b can include primary tabs 106a, 106b (which are the lower tabs before folding, as described below) and secondary tabs 108a, 108b (which are the upper tabs before folding) at an outflow end of the leaflet.

The commissure portions 105a, 105b can also include fold portions 107a, 107b that axially offset the primary tabs 106a, 106b from the secondary tabs 108a, 108b. As shown in FIG. 2, the secondary tabs 108a, 108b can be folded down and positioned adjacent to the primary tabs 106a, 106b by folding the fold portions 107a, 107b of the leaflet 102. When folded, the secondary tabs 108a, 108b and the respective primary tabs 106a, 106b can cooperate to form commissures at the adjacent sides of the leaflets 102. The commissures can be mounted within a frame and in a manner to secure the leaflets to the frame by the commissures. When in the folded configuration, the fold portions 107a, 107b of each leaflet 102 can form inner fold layers 110a, 110b (also referred to as inner layers) and outer fold layers 112a, 112b (also referred to as outer layers) at each commissure. As will be explained further, a retention member can extend between the inner and outer fold layers 110a, 112a of one commissure portion 105a and the inner and outer fold layers 110b, 112b of an adjacent commissure portion 105b. In this way, the retention member can securely hold the commissure portions 105a, 105b of two adjacent leaflets 102 together at each commissure.

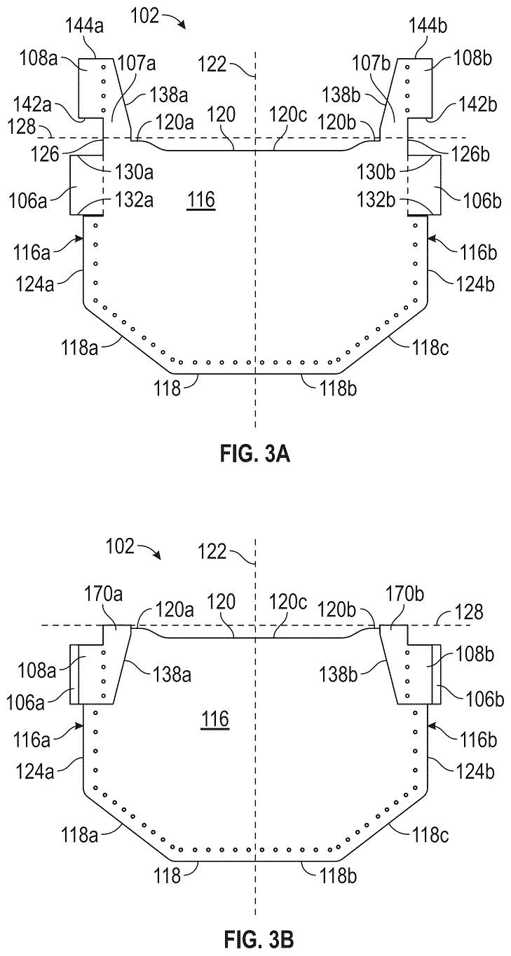

Referring to FIGS. 3A-3B, in one illustrative example, a leaflet 102 includes a body portion 116. The body portion 116 can comprise a flexible sheet of material and can comprise any of the example leaflet materials previously mentioned or known in the art. The body portion 116 includes an inflow edge 118 (lower edge in the figure) and an outflow edge 114 (upper edge in the figure) that is in opposing relation to the inflow edge 118 (also referred to as the cusp edge of the leaflet). When the leaflet 102 is mounted within an annular frame as part of a valvular structure, the inflow edge 118 can be positioned in an inflow end portion of the frame, and the outflow edge 114 can be positioned in an outflow end portion of the frame. The terms “inflow” and “outflow” are related to the normal direction of flow through the frame (for example, forward flow of blood).

The inflow edge 118 may have a truncated V-shape or tapered shape as shown. In one example, the inflow edge 118 may include linear edges 118a, 118b, 118c that are arranged to form the truncated V-shape or tapered shape. In other examples, the inflow edge 118 may have a truncated V-shape or tapered shape composed of one or more curved edges or a combination of linear edges and curved edges. In other implementations, the inflow edge 118 may have a scalloped shape.

In some examples, as depicted, the outflow edge 114 may include riser portions 120a, 120b (also referred to as stepped portions) at opposite ends of the outflow edge 114 and a coaptation portion 120c positioned between the riser portions 120a, 120b. As depicted, top edges of the riser portions 120a, 120b are axially offset from the coaptation portion 120c. In this way, as will be described in more detail below, the riser portions 120a, 120b can offset or elevate a retention member 150 away from the coaptation portion 120c in a final folded configuration, such that the retention member 150 does not interfere with the coaptation portion 120c during working cycles of the leaflet 102. In other examples, the outflow edge 114 may be a linear edge or an approximately linear edge that does not include riser portions 120a, 120b. The approximately linear edge may be formed by two or more linear edges with different inclination angles or by a curved edge with a slight curvature.

The body portion 116 in the illustrated example includes opposite sides 116a, 116b that extend axially between respective ends of the inflow edge 118 and the outflow edge 114. The axial direction of the leaflet 102 is indicated by longitudinal axis 122, which may be an axis that is transverse to the inflow edge 118 and the outflow edge 114. The longitudinal axis 122 may be an axis about which leaflet 102 is symmetrical.

The side 116a includes side edges 124a, 126a, which are spaced apart in the axial direction and are generally parallel to the longitudinal axis 122. The side 116b includes side edges 124b, 126b, which are spaced apart in the axial direction and are generally parallel to the longitudinal axis 122. The side edges 124a, 124b (also referred to as sub-commissure edges of the leaflet) are connected to opposite ends of the inflow edge 118.

During assembly of the valvular structure, the side edges 124a, 124b may be sutured to side edges 124b, 124a of adjacent leaflets (as shown at 104 in FIG. 1) or attached to a frame. In some examples, the side edges 126a, 126b are not sutured to side edges 126b, 126a of adjacent leaflets nor attached to a frame. Instead, the side edges 126a, 126b can be held together with the side edges 126b, 126a of an adjacent leaflet by a retention member 150 (for example, when the leaflet 102 is in a folded configuration, etc.), as described below.

The leaflet 102 may be attached to a frame (for example, to struts of the frame) along the inflow edge 118. For example, the inflow edge 118 can be connected to a frame at an inflow end portion of the frame. The outflow edge 114 can be free to move relative to the frame. For example, the outflow edge 114 can move radially inward and outward during a working cycle of the leaflet 102.

The leaflet 102 has opposite primary tabs 106a, 106b (lower tabs in the figure) projecting from opposite sides 116a, 116b of the body portion 116. The primary tabs 106a, 106b are involved in forming commissures of the valvular structure as previously described. The primary tab 106a extends between the side edges 124a, 126a. The primary tab 106b extends between the side edges 124b, 126b. The primary tabs 106a, 106b have first edges 130a, 130b (top edges in the figure) and second edges 132a, 132b (bottom edges in the figure).

The side edges 126a, 126b extend along an outer edge of fold portions 107a, 107b in the axial direction. The fold portions 107a, 107b have inner edges 138a, 138b on opposite sides as the side edges 126a, 126b. The inner edges 138a, 138b extend along the length (or height) of the fold portions 107a, 107b in the axial direction. In some examples, as depicted, the inner edges 138a, 138b are angled relative to the longitudinal axis 122.

The leaflet 102 has secondary tabs 108a, 108b (upper tabs in the figure) extending from the fold portions 107a, 107b, respectively. Specifically, the secondary tab 108a, 108b are offset from the primary tabs 106a, 106b in the axial direction by the length (or height) of the side edges 126a, 126b. The secondary tab 108a can be arranged orthogonally to the fold portion 107a to form an L shape. Similarly, the secondary tab 108b can be arranged orthogonally to the fold portion 107b to form an L shape. The secondary tabs 108a, 108b have first edges 142a, 142b (bottom edges in the figure) and second edges 144a, 144b (top edges in the figure).

The secondary tab 108a is located on the same side of the leaflet 102 as the primary tab 106a (left side in the figure) and forms a cooperating pair with the primary tab 106a, such that tabs 108a, 106a will work together to form a commissure with an adjacent pair of primary and secondary tabs of an adjacent leaflet. The secondary tab 108b is located on the same side of the leaflet 102 as the primary tab 106b (right side in the figure) and forms a cooperating pair with the primary tab 106b, such that tabs 108b, 106b will work together to form a commissure with an adjacent pair of primary and secondary tabs of an adjacent leaflet.

When forming the valvular structure, the secondary tabs 108a, 108b can be folded about a horizontal fold line 128 to place the secondary tab 108a against or adjacent to the primary tab 106a and the secondary tab 108b against or adjacent to the primary tab 106b. In one example, the fold line 128 is positioned halfway between the first edges 130a, 130b of the primary tabs 106a, 106b and the first edges 142a, 142b of the second tab portions 108a, 108b, such that the fold line 128 is coincident or aligned with an axial midline of the side edges 126a, 126b, as shown in FIG. 3. In some cases, the secondary tabs 108a, 108b may be folded against the primary tabs 106a, 106b by matching the first edges 142a, 142b of the secondary tabs 108a, 108b with the first edges 130a, 130b of the primary tabs 106a, 106b, respectively.

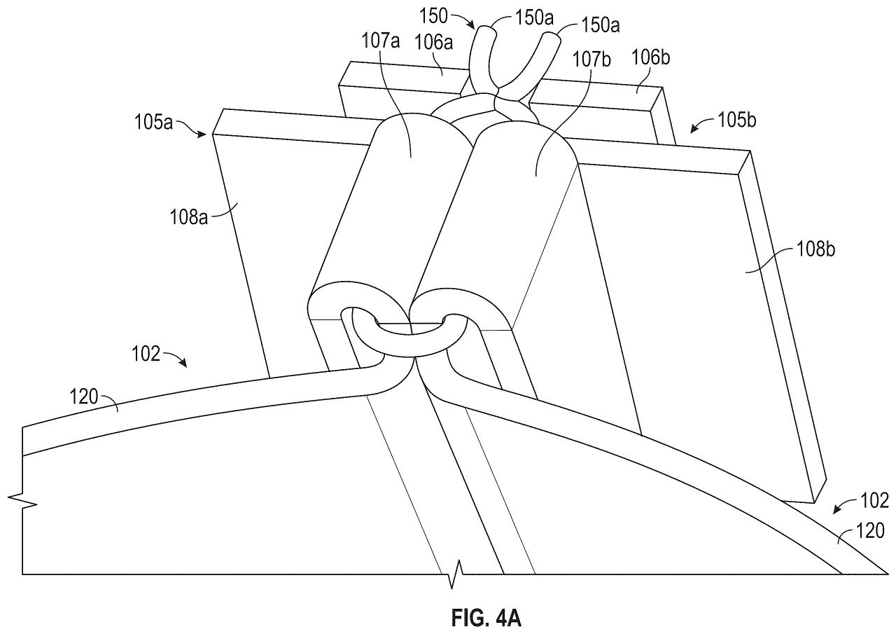

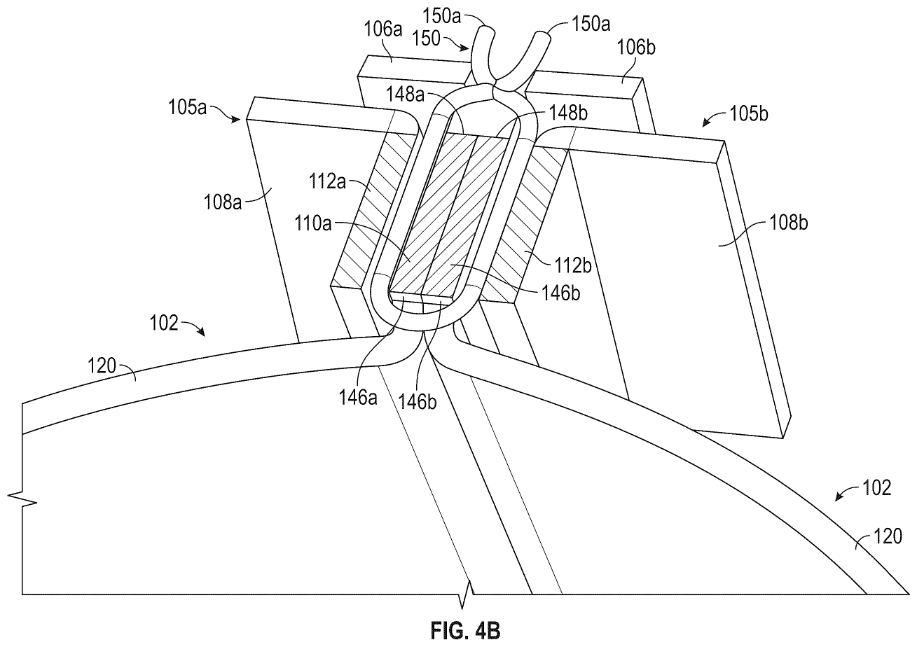

FIGS. 4A and 4B show a top perspective view of a commissure formed by two adjacent leaflets 102 in a folded configuration. As shown in FIGS. 4A and 4B, a retention member 150 can extend around the commissure portions 105a, 105b of the two adjacent leaflets 102 to hold the adjacent commissure portions 105a, 105b together during working cycles of a prosthetic valve (for example, valve 300 of FIG. 6A, discussed below). Specifically, the retention member 150 can extend between the inner and outer fold layers 110a, 112a of one commissure portion 105a and the inner and outer fold layers 110b, 112b of an adjacent commissure portion 105b. A portion of the fold portions 107a, 107b is removed from FIG. 4B for purposes of illustration.

The retention member 150 (also referred to herein as a retention ring or loop) can be configured to securely hold the fold portions 107a, 107b together at each commissure. The retention member 150 can prevent the fold portions 107a, 107b from separating circumferentially away from each other (for example, preventing a V-shaped separation) during working cycles of the valvular structure 100 (for example, under the forward flow of blood). Specifically, the retention member 150 can prevent separation of the inner layers 110a, 110b, which can prevent or minimize contact between the free edges of the leaflets and the frame under the forward flow of blood.

The retention member 150 can be coupled to a pair of adjacent leaflets 102 at the commissure such that the retention member 150 and/or other mechanisms for attachment (for example, stitches, etc.) do not pierce or penetrate any portions of the leaflets 102. For example, the retention member 150 can be configured to securely hold the leaflets 102 together at the commissures without requiring any stitches or other forms of invasive attachment to couple the retention member 150 to the leaflets 102. Additionally, the retention member 150 can be configured to securely hold the fold portions 107a, 107b of adjacent leaflets 102 against each other without the retention member 150 piercing through the leaflets 102 (for example, without the retention member 150 being threaded through the commissure portions 105a, 105b, etc.). As such, the leaflets 102 can remain intact at their commissure portions 105a, 105b.

The retention member 150 can comprise a fully enclosed loop, band, or ring having, for example, a circular or elliptical shape. In some examples, the retention member 150 can made of a flexible and/or compliant material. For example, as depicted, the retention member 150 can comprise a flexible suture or cord having both ends 150a tied together to form a loop. As shown, the ends 150a can be positioned radially outward relative to the commissure to prevent the ends 150a from interfering with the outflow edge 114 of the leaflets 102 during working cycles. In other examples, the retention member 150 can comprise a loop that is closed in other manners (for example, by connecting the ends of the retention member with a separate coupling element, etc.). In other examples, the retention member 150 can be pre-formed (for example, molded) as a continuous band or ring that does not have any free ends that need to be tied or otherwise connected to each other. In instances where the retention member 150 is pre-formed as a continuous band or ring, the secondary tabs 108a, 108b can be inserted through the retention member 150 and subsequently be folded around the retention member 150 and against the primary tabs 106a, 106b during assembly of the valvular structure.

The retention member 150 can be made from materials designed to resist tissue ingrowth. For example, the retention member 150 can be made from Teflon, thermoplastic polyurethane (TPU), pericardial tissue (or any other tissue) and the like. In some examples, the retention member 150 can be made from a relatively more rigid material, such as a metal wire (for example, a wire can include Nitinol, stainless steel or a cobalt-chromium alloy).

In still other examples, the retention member 150 can be an open or non-continuous band or ring that has free ends that are not connected to each other, although the retention member has sufficient rigidity to resist radial expansion under normal working pressures and therefore can function prevent separation of the inner layers 110a, 110b of a commissure. In such examples, the retention member can be made of a relatively rigid material, such as a metal wire (for example, a wire can include Nitinol, stainless steel or a cobalt-chromium alloy). The free ends of the retention member can be positioned radially outward of the commissure tabs and/or the frame of the prosthetic valve, such as in the vicinity of the ends 150a, 150b of the retention member shown in FIG. 4B.

In some examples, as depicted, when tabs of the leaflets 102 are folded, a narrow channel or pathway is formed by inner surfaces of the fold portions 107a, 107b (for example, beneath top segments of the fold portions 107a, 107b) and the retention member 150 can extend through the channel. As shown in FIG. 4B, the retention member 150 can extend continuously in a closed loop through the inner and outer fold layers 110a, 112a of the commissure portion 105a, around radial inner edges 146a, 146b of the inner fold layers 110a, 110b, through the inner and outer fold layers 110b, 112b of the second commissure portion 105b and around radial outer edges 148a, 148b of the inner fold layers 110a, 110b. As such, the retention member 150 can encircle the inner fold layers 110a, 110b of the commissure portions 105a, 105b.

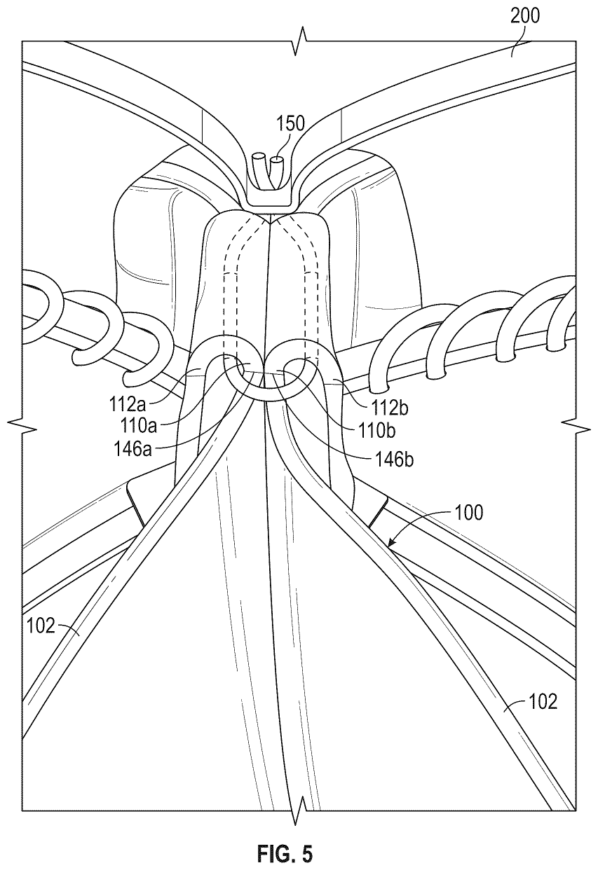

The retention member 150 can be placed in position during commissure assembly, prior to attachment of the valvular structure 100 to a frame 200. FIG. 5 shows the valvular structure 100 in an assembled state, that is, with the valvular structure 100 mounted within a frame 200. In the assembled state, as depicted, the retention member 150 extends radially relative to the frame 200 and is positioned radially inward of the frame 200. The retention member 150 also can be positioned in a horizontal plane that is parallel to the fold line 128 and perpendicular to the longitudinal axis 122 of the leaflet 102 (FIGS. 3A-3B). In other examples, the retention member 150 can be disposed at an angle greater than 0 degrees relative to the horizontal plane and the fold line 128.

As shown in FIG. 5, when the secondary tabs 108a, 108b are folded against the primary tabs 106a, 106b, the fold portions 107a, 107b can form a double layer of leaflet material at the commissures. The outer fold layers 112a, 112b are positioned flat and adjacent to and/or abutting the inner fold layers 110a, 110b, such that each commissure comprises four layers of leaflet material. When attached to the frame 200, the four layers of leaflet material can be positioned just inside of the frame 200 (for example, radially inside of a commissure window). This four layered portion of the commissures can be more resistant to bending, or articulating, than the portion of the leaflets 102 just radially inward from the relatively more rigid four layered portion. This causes the leaflets 102 to articulate primarily at inner edges 146a, 146b of the inner fold layers 110a, 110b in response to blood flowing through the valve during operation within the body, as opposed to articulating about the frame. Because the leaflets articulate at a location spaced radially inwardly from the frame, the leaflets can avoid contact with and damage from the frame. The retention member 150 can be configured to prevent the inner fold layers 110a, 110b of the commissures from splaying apart in a circumferential direction, for example, under high forces during working cycles of the valve. Additionally, the retention member 150 can help ensure that the leaflets 102 articulate primarily at the radially inner edges 146a, 146b during valve cycling.

During a working cycle of the valvular structure 100, the leaflets 102 can experience various pressures including, for example, pressures at attachment locations between the leaflets 102 and the frame 200. Specifically, the primary tabs 106a, 106b of the leaflet 102 can be coupled to commissure windows of the frame 200, as described in more detail below. The primary tabs 106a, 106b can experience relatively high pressures under the reverse flow of blood (for example, when the leaflets 102 are closed, during systole, etc.).

A pressure gradient exists between the primary tabs 106a, 106b and the location where the retention member 150 is coupled to the leaflets 102. For example, when the leaflets 102 are closed, the primary tabs 106a, 106b can experience relatively high pressures and the inner layers 110a, 110b (around which the retention member 150 extends) can experience relatively negligible pressures. This pressure gradient is due in part to the geometry of the leaflet 102, including the axial spacing between the primary tabs 106a, 106b and the top of the inner layers 110a, 110b (at the fold line 128, as shown in FIG. 3B). Due to the axial distance between the primary tabs 106a, 106b and the retention member 150, the retention member 150 is elevated axially away from the higher-pressure location of the leaflets 102 when the leaflets 102 are closed. In this way, the retention member 150 can experience relatively negligible pressures under the reverse flow of blood as compared to the primary tabs 106a, 106b which carry a majority of the load of the commissures. Advantageously, the retention member 150 can resist separation of the inner layers 110a, 110b during the forward flow of blood while avoiding over-stressing the leaflets during the reverse flow of blood.

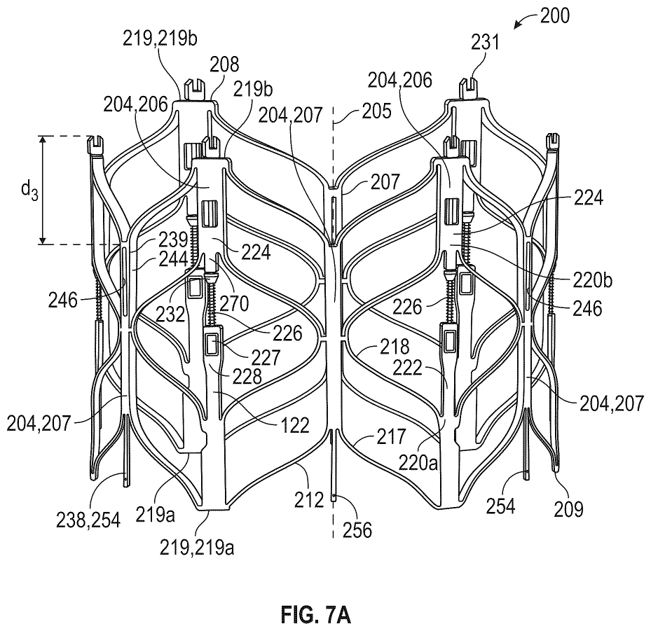

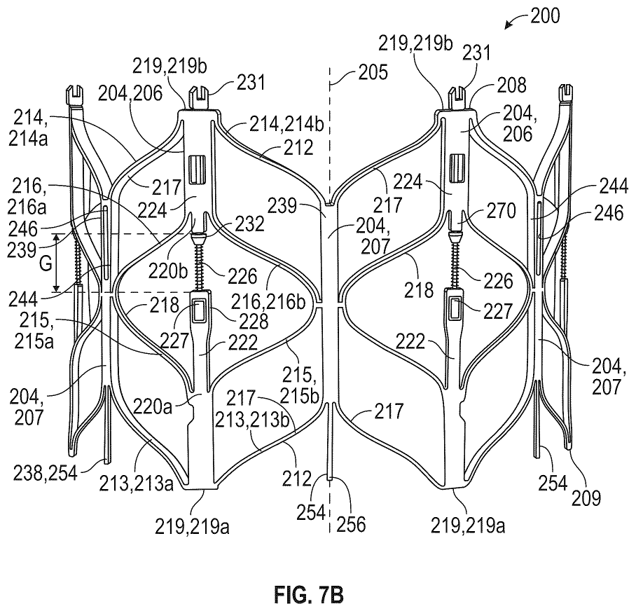

FIGS. 6A-7B illustrate an example of a prosthetic valve 300 (which also may be referred to herein as “prosthetic heart valve 300”) having a frame 200. FIGS. 7A-7B show the frame 200 by itself, while FIGS. 6A-6B show the frame 200 with the valvular structure 100 mounted within and to the annular frame 200. FIG. 6B additionally shows an optional skirt assembly comprising an outer skirt 303. While only one side of the frame 200 is depicted in FIG. 7B, it should be appreciated that the frame 200 forms an annular structure having an opposite side that is substantially identical to the portion shown in FIG. 6B, as shown in FIGS. 6A-7A.

As shown in FIGS. 6A and 6B, the valvular structure 100 is coupled to and supported inside the frame 200. The valvular structure 100 is configured to regulate the flow of blood through the prosthetic valve 300, from an inflow end portion 334 to an outflow end portion 336. The valvular structure 100 is shown in a fully assembled state with commissures 152 that couple the leaflets 102 to the frame 200. Specifically, each commissure 152 can be secured to a respective commissure support structure 244 (also referred to herein as “commissure supports”) and/or to other portions of the frame 200, as described in greater detail below.

The prosthetic valve 300 may include one or more skirts mounted around the frame 200. For example, as shown in FIG. 6B, the prosthetic valve 300 may include an outer skirt 303 mounted around an outer surface of the frame 200. The outer skirt 303 can function as a sealing member for the prosthetic valve 300 by sealing against the tissue of the native valve annulus and helping to reduce paravalvular leakage past the prosthetic valve 300. In some cases, an inner skirt (not shown) may be mounted around an inner surface of the frame 200. The inner skirt can function as a sealing member to prevent or decrease perivalvular leakage, to anchor the leaflets 102 to the frame 200, and/or to protect the leaflets 102 against damage caused by contact with the frame 200 during crimping and during working cycles of the prosthetic valve 300. In some examples, the inflow edge portions 118 of the leaflets 102 can be sutured to the inner skirt generally along the scallop line. The inner skirt can in turn be sutured to adjacent struts 212 of the frame 200. In other examples, as shown in FIG. 6A, the leaflets 102 can be sutured directly to the frame 200 or to a reinforcing member 325 (also referred to as a reinforcing skirt or connecting skirt) in the form of a strip of material (for example, a fabric strip) which is then sutured to the frame 200, along the scallop line via stitches (for example, whip stitches) 333.

The inner and outer skirts and the connecting skirt 325 can include any of various suitable biocompatible materials, including any of various synthetic materials, including fabrics (for example, polyethylene terephthalate fabric) or natural tissue (for example, pericardial tissue). Further details regarding the use of skirts or sealing members in prosthetic valve can be found, for example, in U.S. Patent Publication No. 2020/0352711, which is incorporated herein by reference.

The frame 200, which is shown alone and in greater detail in FIGS. 7A and 7B, comprises and inflow end 209, an outflow end 208, and a plurality of axially extending posts 204. The axial direction of the frame 200 is indicated by a longitudinal axis 205, which extends from the inflow end 209 to the outflow end 208 (FIGS. 6A and 6B). Some of the posts 204 can be arranged in pairs of axially aligned first and second struts or posts 222, 224. An actuator 226 (such as the illustrated threaded rod or bolt) can extend through one or more pairs of posts 222, 224 to form an integral expansion and locking mechanism or actuator mechanism 206 configured to radially expand and compress the frame 200, as further described below. One or more of posts 204 can be configured as support posts 207.

The actuator mechanisms 206 (which can be used to radially expand and/or radially compress the prosthetic valve 300) can be integrated into the frame 200 of the prosthetic valve 300, thereby reducing the crimp profile and/or bulk of the prosthetic valve 300. Integrating the actuator mechanisms 206 (which can also be referred to herein as “expansion and locking mechanisms”) into the frame 200 can also simplify the design of the prosthetic valve 300, making the prosthetic valve 300 less costly and/or easier to manufacture. In the illustrated example, an actuator 226 extends through each pair of axially aligned posts 222, 224. In other examples, one or more of the pairs of posts 222, 224 can be without a corresponding actuator.

The posts 204 can be coupled together by a plurality of circumferentially extending link members or struts 212. Each strut 212 extends circumferentially between adjacent posts 204 to connect all of the axially extending posts 204. As one example, the prosthetic valve 300 can include equal numbers of support posts 207 and pairs of actuator posts 222, 224 and the pairs of posts 222, 224 and the support posts 207 can be arranged in an alternating order such that each strut 212 is positioned between one of the pairs of posts 222, 224 and one of the support posts 207 (that is, each strut 212 can be coupled on one end to one of the posts 222, 224 and can be coupled on the other end to one of the support posts 207). However, the prosthetic valve 300 can include different numbers of support posts 207 and pairs of posts 222, 224 and/or the pairs of posts 222, 224 and the support posts 207 can be arranged in a non-alternating order, in other examples.

As illustrated in FIG. 7B, the struts 212 can include a first row of struts 213 at or near the inflow end 209 of the prosthetic valve 300, a second row of struts 214 at or near the outflow end 208 of the prosthetic valve 300, and third and fourth rows of struts 215, 216, respectively, positioned axially between the first and second rows of struts 213, 214. The struts 212 can form and/or define a plurality of cells (that is, openings) in the frame 200. For example, the struts 213, 214, 215, and 216 can at least partially form and/or define a plurality of first cells 217 and a plurality of second cells 218 that extend circumferentially around the frame 200. Specifically, each first cell 217 can be formed by two struts 213a, 213b of the first row of struts 213, two struts 214a, 214b of the second row of struts 214, and two of the support posts 207. Each second cell 218 can be formed by two struts 215a, 115b of the third row of struts 215 and two struts 216a, 216b of the fourth row of struts 216. As illustrated in FIGS. 7A and 7B, each second cell 218 can be disposed within one of the first cells 217 (that is, the struts 215a-716b forming the second cells 218 are disposed between the struts forming the first cells 217 (that is, the struts 213a, 213b and the struts 214a, 214b), closer to an axial midline of the frame 200 than the struts 213a-214b).

As illustrated in FIGS. 7A and 7B, the struts 212 of frame 200 can comprise a curved shape. Each first cell 217 can have an axially-extending hexagonal shape including first and second apices 219 (for example, an inflow apex 219a and an outflow apex 219b). In examples where the delivery apparatus is releasably connected to the outflow apices 219b (as described below), each inflow apex 219a can be referred to as a “distal apex” and each outflow apex 219b can be referred to as a “proximal apex”. Each second cell 218 can have a diamond shape including first and second apices 220 (for example, distal apex 220a and proximal apex 220b). In some examples, the frame 200 comprises six first cells 217 extending circumferentially in a row, six second cells 218 extending circumferentially in a row within the six first cells 217, and twelve posts 204. However, in other examples, the frame 200 can comprise a greater or fewer number of first cells 217 and a correspondingly greater or fewer number of second cells 218 and posts 204.

As noted above, some of the posts 204 can be arranged in pairs of first and second posts 222, 224. The posts 222, 224 are aligned with each other along the length of the frame 200 and are axially separated from one another by a gap G (FIG. 7B) (those with actuators 226 can be referred to as actuator posts or actuator struts). Each first post 222 (that is, the lower post shown in FIGS. 7A and 7B) can extend axially from the inflow end 209 of the prosthetic valve 300 toward the second post 224, and the second post 224 (that is, the upper post shown in FIGS. 7A and 7B) can extend axially from the outflow end 208 of the prosthetic valve 300 toward the first post 222. For example, each first post 222 can be connected to and extend from an inflow apex 219a and each second post 224 can be connected to and extend from an outflow apex 219b. Each first post 222 and the second post 224 can include an inner bore configured to receive a portion of an actuator member, such as in the form of a substantially straight threaded rod 226 (or bolt) as shown in the illustrated example. The threaded rod 226 also may be referred to herein as actuator 226, actuator member 226, and/or screw actuator 226. In examples where the delivery apparatus can be releasably connected to the outflow end 208 of the frame 200, the first posts 222 can be referred to as distal posts or distal axial struts and the second posts 224 can be referred to as proximal posts or proximal axial struts.

Each threaded rod 226 extends axially through a corresponding first post 222 and second post 224. Each threaded rod 226 also extends through a bore of a nut 227 captured within a slot or window formed in an end portion 228 of the first post 222. The threaded rod 226 has external threads that engage internal threads of the bore of the nut 227. The inner bore of the second post 224 (through which the threaded rod 226 extends) can have a smooth and/or non-threaded inner surface to allow the threaded rod 226 to slide freely within the bore. Rotation of the threaded rod 226 relative to the nut 227 produces radial expansion and compression of the frame 200, as further described below.

In some examples, the threaded rod 226 can extend past the nut 227 toward the inflow end 209 of the frame 200 into the inner bore of the first post 222. The nut 227 can be held in a fixed position relative to the first post 222 such that the nut 227 does not rotate relative to the first post 222. In this way, whenever the threaded rod 226 is rotated (for example, by a physician) the threaded rod 226 can rotate relative to both the nut 227 and the first post 222. The engagement of the external threads of the threaded rod 226 and the internal threads of the nut 227 prevent the rod 226 from moving axially relative to the nut 227 and the first post 222 unless the threaded rod 226 is rotated relative to the nut 227. Thus, the threaded rod 226 can be retained or held by the nut 227 and can only be moved relative to the nut 227 and/or the first post 222 by rotating the threaded rod 226 relative to the nut 227 and/or the first post 222. In other examples, in lieu of using the nut 227, at least a portion of the inner bore of the first post 222 can be threaded. For example, the bore along the end portion 228 of the first post 222 can comprise inner threads that engage the external threaded rod 226 such that rotation of the threaded rod causes the threaded rod 226 to move axially relative to the first post 222.

When a threaded rod 226 extends through and/or is otherwise coupled to a pair of axially aligned posts 222, 224, the pair of axially aligned posts 222, 224 and the threaded rod 226 can serve as one of the expansion and locking mechanisms 206. In some examples, a threaded rod 226 can extend through each pair of axially aligned posts 222, 224 so that all of the posts 222, 224 (with their corresponding rods 226) serve as expansion and locking mechanisms 206. As just one example, the prosthetic valve 300 can include six pairs of posts 222, 224, and each of the six pairs of posts 222, 224 with their corresponding rods 226 can be configured as one of the expansion and locking mechanisms 206 for a total of six expansion and locking mechanisms 206. In other examples, not all pairs of posts 222, 224 need be expansion and locking mechanisms (that is, actuators). If a pair of posts 222, 224 is not used as an expansion and locking mechanism, a threaded rod 226 need not extend through the posts 222, 224 of that pair.

The threaded rod 226 can be rotated relative to the nut 227, the first post 222, and the second post 224 to axially foreshorten and/or axially elongate the frame 200, thereby radially expanding and/or radially compressing, respectively, the frame 200 (and therefore the prosthetic valve 300). Specifically, when the threaded rod 226 is rotated relative to the nut 227, the first post 222, and the second post 224, the first and second posts 222, 224 can move axially relative to one another, thereby widening or narrowing the gap G (FIG. 7B) separating the posts 222, 224, and thereby radially compressing or radially expanding the prosthetic valve 300, respectively. Thus, the gap G (FIG. 7B) between the first and second posts 222, 224 narrows as the frame 200 is radially expanded and widens as the frame 200 is radially compressed.

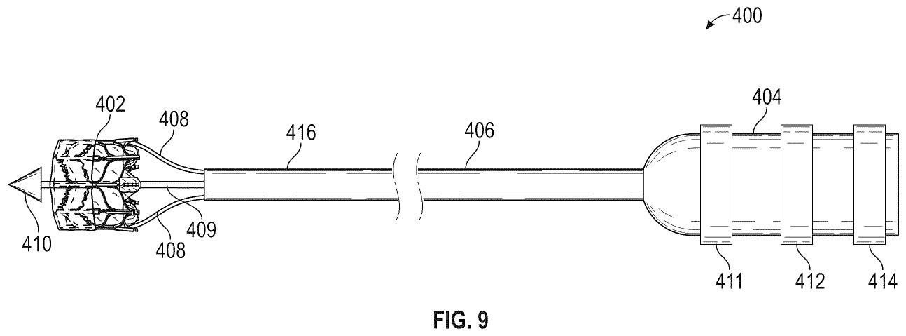

The threaded rod 226 can extend proximally past the proximal end of the second post 224 and can include a head portion 231 at its proximal end that can serve at least two functions. First, the head portion 231 can removably or releasably couple the threaded rod 226 to a respective actuator assembly of a delivery apparatus that can be used to radially expand and/or radially compress the prosthetic valve 300 (for example, the delivery apparatus 400 of FIG. 9, as described below). Second, the head portion 231 can prevent the second post 224 from moving proximally relative to the threaded rod 226 and can apply a distally directed force to the second post 224, such as when radially expanding the prosthetic valve 300. Specifically, the head portion 231 can have a width greater than a diameter of the inner bore of the second post 224 such that the head portion 231 is prevented from moving into the inner bore of the second post 224. Thus, as the threaded rod 226 is threaded farther into the nut 227, the head portion 231 of the threaded rod 226 draws closer to the nut 227 and the first post 222, thereby drawing the second post 224 towards the first post 222, and thereby axially foreshortening and radially expanding the prosthetic valve 300.

The threaded rod 226 also can include a stopper 232 (for example, in the form of a nut, washer or flange) disposed thereon. The stopper 232 can be disposed on the threaded rod 226 such that it sits within the gap G. Further, the stopper 232 can be integrally formed on or fixedly coupled to the threaded rod 226 such that it does not move relative to the threaded rod 226. Thus, the stopper 232 can remain in a fixed axial position on the threaded rod 226 such that it moves in lockstep with the threaded rod 226.

Rotation of the threaded rod 226 in a first direction (for example, clockwise) can cause corresponding axial movement of the first and second posts 222, 224 toward one another, thereby decreasing the gap G and radially expanding the frame 200, while rotation of the threaded rod 226 in an opposite second direction causes corresponding axial movement of the first and second posts 222, 224 away from one another, thereby increasing the gap G and radially compressing the frame. When the threaded rod 226 is rotated in the first direction, the head portion 231 of the rod 226 bears against an adjacent surface of the frame (for example, an outflow apex 219b), while the nut 227 and the first post 222 travel proximally along the threaded rod 226 toward the second post 224, thereby radially expanding the frame. As the frame 200 moves from a compressed configuration to an expanded configuration, the gap G between the first and second posts 222, 224 can narrow.

When the threaded rod 226 is rotated in the second direction, the threaded rod 226 and the stopper 232 move toward the outflow end 208 of the frame until the stopper 232 abuts the inflow end 270 of the second post 224 (as shown in FIGS. 7A and 7B). Upon further rotation of the rod 226 in the second direction, the stopper 232 can apply a proximally directed force to the second post 224 to radially compress the frame 200. Specifically, during crimping/radial compression of the prosthetic valve 300, the threaded rod 226 can be rotated in the second direction (for example, counterclockwise) causing the stopper 232 to push against (that is, provide a proximally directed force to) the inflow end 270 of the second post 224, thereby causing the second post 224 to move away from the first post 222, and thereby axially elongating and radially compressing the prosthetic valve 300.

Thus, each of the second posts 224 can slide axially relative to a corresponding one of the first posts 222 but can be axially retained and/or restrained between the head portion 231 of a threaded rod 226 and a stopper 232. That is, each second post 224 can be restrained at its proximal end by the head portion 231 of the threaded rod 226 and at its distal end by the stopper 232. In this way, the head portion 231 can apply a distally directed force to the second post 224 to radially expand the prosthetic valve 300 while the stopper 232 can apply a proximally directed force to the second post 224 to radially compress the prosthetic valve 300. As explained above, radially expanding the prosthetic valve 300 axially foreshortens the prosthetic valve 300, causing an inflow end portion 334 and outflow end portion 336 of the prosthetic valve 300 (FIGS. 6A and 6B) to move towards one another axially, while radially compressing the prosthetic valve 300 axially elongates the prosthetic valve 300, causing the inflow and outflow end portions 334, 336 to move away from one another axially.

In other examples, the threaded rod 226 can be fixed against axial movement relative to the second post 224 (and the stopper 232 can be omitted) such that rotation of the threaded rod 226 in the first direction produces proximal movement of the nut 227 and radial expansion of the frame 200 and rotation of the threaded rod 226 in the second direction produces distal movement of the nut 227 and radial compression of the frame 200.

As also introduced above, some of the posts 204 can be configured as support posts 207. As shown in FIGS. 7A and 7B, the support posts 207 can extend axially between the inflow and outflow ends 209, 208 of the frame 200 and each can have an inflow end portion 238 and an outflow end portion 239. The outflow end portion 239 of one or more support posts 207 can include a commissure support structure or member 244. The commissure support structure 244 can comprise strut portions defining a commissure opening 246 therein.

The commissure opening 246 (which can also be referred to herein as a “commissure window 246”) can extend radially through a thickness of the support post 207 and can be configured to accept a portion of a valvular structure 100 (for example, a commissure 152) to couple the valvular structure 100 to the frame 200. For example, each commissure 152 can be mounted to a respective commissure support structure 244, such as by inserting a pair of commissure tabs of adjacent leaflets 102 through the commissure opening 246 and suturing the commissure tabs to each other and/or the commissure support structure 244. In some examples, the commissure opening 246 can be fully enclosed by the support post 207 such that a portion of the valvular structure 100 can be slid radially through the commissure opening 246, from an interior to an exterior of the frame 200, during assembly. In the illustrated example, the commissure opening 246 has a substantially rectangular shape that is shaped and sized to receive commissure tabs of two adjacent leaflets therethrough. However, in other examples, the commissure opening can have any of various shapes (for example, square, oval, square-oval, triangular, L-shaped, T-shaped, C-shaped, etc.).

The commissure openings 246 are spaced apart about the circumference of frame 200 (or angularly spaced apart about frame 200). The spacing may or may not be even. In one example, the commissure openings 246 are axially offset from the outflow end 208 of the frame 200 by an offset distance d₃ (indicated in FIG. 7A). As an example, the offset distance d₃ may be in a range from 2 mm to 6 mm. In general, the offset distance d₃ should be selected such that when the leaflets are attached to the frame 200 via the commissure openings 246, the free edge portions (for example, outflow edge portions) of the leaflets 102 will not protrude from or past the outflow end 208 of the frame 200.

The frame 200 can comprise any number of support posts 207, any number of which can be configured as commissure support structures 244. For example, the frame 200 can comprise six support posts 207, three of which are configured as commissure support structures 244. However, in other examples, the frame 200 can comprise more or less than six support posts 207 and/or more or less than three commissure support structures 244.

The inflow end portion 238 of each support post 207 can comprise an extension 254 (show as a cantilevered strut in FIGS. 7A and 7B) that extends toward the inflow end 209 of the frame 200. Each extension 254 can comprise an aperture 256 extending radially through a thickness of the extension 254. In some examples, the extension 254 can extend such that an inflow edge of the extension 254 aligns with or substantially aligns with the inflow end 209 of the frame 200. In use, the extension 254 can prevent or mitigate portions of an outer skirt from extending radially inwardly and thereby prevent or mitigate any obstruction of flow through the frame 200 caused by the outer skirt. The extensions 254 can further serve as supports to which portions of the inner and/or outer skirts and/or the leaflets and/or the connecting skirt 325 can be coupled. For example, sutures used to connect the inner and/or outer skirts and/or the leaflets and/or the connecting skirt 325 can be wrapped around the extensions 254 and/or can extend through apertures 256.

As an example, each extension 254 can have an aperture 256 (FIG. 7A) or other features to receive a suture or other attachment material for connecting an adjacent inflow edge portion 118 of a leaflet 102 (FIG. 6A), the outer skirt 303 (in FIG. 6B), the connecting skirt 325, and/or an inner skirt. In some examples, the inflow edge portion 118 of each leaflet 102 can be connected to a corresponding extension via a suture 335 (FIG. 6A).

In some examples, the outer skirt 303 can be mounted around the outer surface of frame 200 as shown in FIG. 6B and the inflow edge of the outer skirt 303 (lower edge in FIG. 6B) can be attached to the connecting skirt 325 and/or the inflow edge portions 118 of the leaflets 102 that have already been secured to frame 200 as well as to the extensions 254 of the frame by sutures 329. The outflow edge of the outer skirt 303 (the upper edge in FIG. 6B) can be attached to selected struts with stitches 337. In implementations where the prosthetic valve includes an inner skirt, the inflow edge of the inner skirt can be secured to the inflow edge portions 118 before securing the cusp edge portions to the frame so that the inner skirt will be between the leaflets and the inner surface of the frame. After the inner skirt and leaflets are secured in place, then the outer skirt can be mounted around the frame as described above.

Further details regarding transcatheter prosthetic heart valves including the manner in which the valvular structure can be mounted to the frame of the prosthetic valve can be found, for example, in U.S. Pat. Nos. 6,730,118, 7,393,360, 7,510,575, 7,993,394, and 8,252,202, U.S. Publication No. 2018/0325665, and U.S. Provisional Application No. 62/854,702, filed May 30, 2019, all of which are incorporated herein by reference in their entireties.

The frame 200 can be a unitary and/or fastener-free frame that can be constructed from a single piece of material (for example, Nitinol, stainless steel or a cobalt-chromium alloy), such as in the form of a tube. The plurality of cells can be formed by removing portions (for example, via laser cutting) of the single piece of material. The threaded rods 226 can be separately formed and then be inserted through the bores in the second (proximal) posts 224 and threaded into the threaded nuts 227.

In some examples, the frame 200 can include a plastically-expandable material, such as stainless steel or a cobalt-chromium alloy. When the frame includes a plastically-expandable material, the prosthetic valve 300 can be placed in a radially compressed state along the distal end portion of a delivery apparatus for insertion into a patient's body. When at the desired implantation site, the frame 200 (and therefore the prosthetic valve 300) can be radially expanded from the radially compressed state to a radially expanded state via actuation of actuation assemblies of the delivery apparatus (as further described below), which rotate the rods 226 to produce expansion of the frame 200. During delivery to the implantation site, the prosthetic valve 300 can be placed inside of a delivery capsule (sheath) to protect against the prosthetic valve contacting the patient's vasculature, such as when the prosthetic valve is advanced through a femoral artery. The capsule can also retain the prosthetic valve in a compressed state having a slightly smaller diameter and crimp profile than may be otherwise possible without a capsule by preventing any recoil (expansion) of the frame once it is crimped onto the delivery apparatus.

In other examples, the frame 200 can include a self-expandable material (for example, Nitinol). When the frame 200 includes a self-expandable material, the prosthetic valve can be radially compressed and placed inside the capsule of the delivery apparatus to maintain the prosthetic valve in the radially compressed state while it is being delivered to the implantation site. When at the desired implantation site, the prosthetic valve is deployed or released from the capsule. In some examples, the frame (and therefore the prosthetic valve) can partially self-expand from the radially compressed state to a partially radially expanded state. The frame 200 (and therefore the prosthetic valve 300) can be further radially expanded from the partially expanded state to a further radially expanded state via actuation of actuation assemblies of the delivery apparatus (as further described below), which rotate the rods 226 to produce expansion of the frame.

Further details of the construction and function of the frame 200 can be found in WIPO Publication No. WO2022/072564, which is incorporated herein by reference.

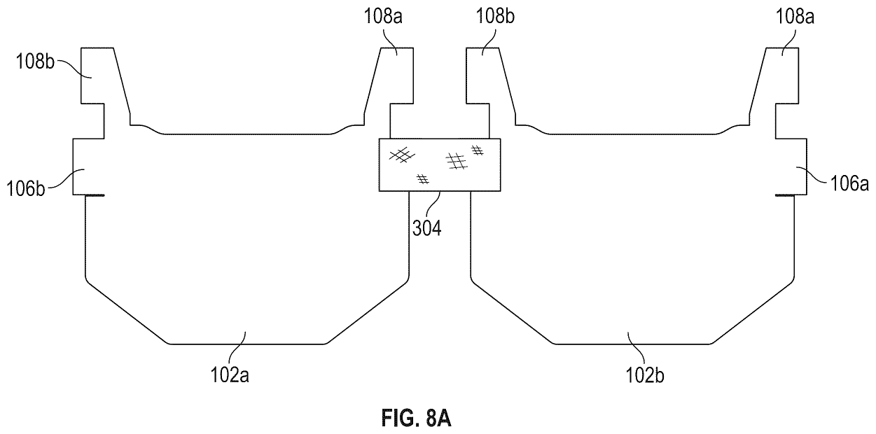

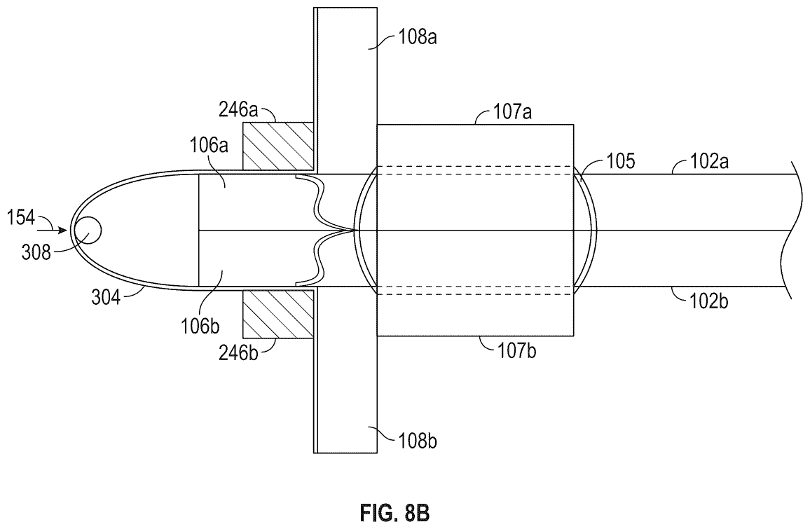

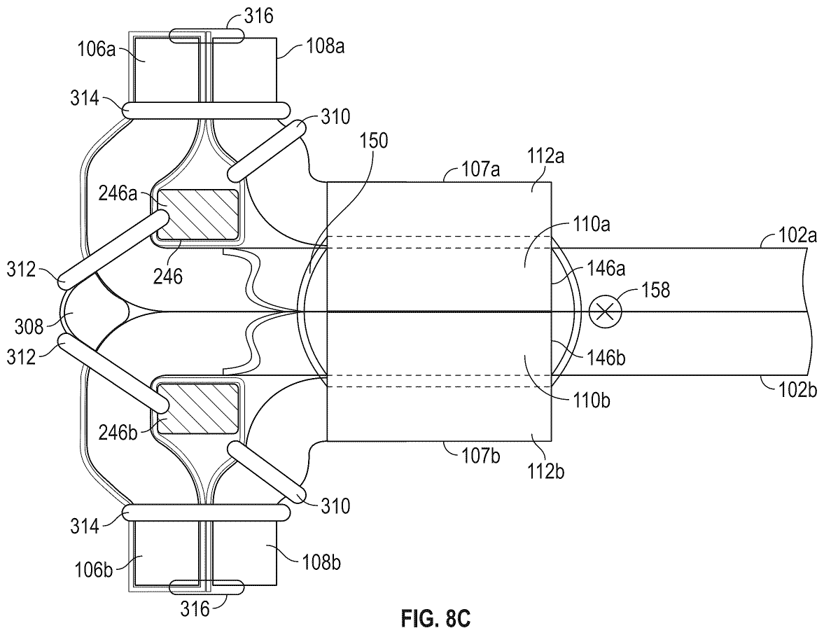

Referring to FIG. 8A, in particular examples, a leaflet assembly or valvular structure can be formed by connecting a flexible connector 304 to a pair of leaflets 102a, 102b at a primary tab 106a of the leaflet 102a and a primary tab 106b of the leaflet 102b. The flexible connector 304 can be connected to the primary tabs 106a, 106b with sutures. The flexible connector 304 can comprise, for example, a piece of fabric (for example, PET fabric). A wedge element 308 (FIGS. 8B and 8C) can be connected to one side of the flexible connector 304. The wedge element 308 can comprise, for example, a relatively heavy gauge suture, such as a braided suture (for example, an Ethibond suture), or a piece of fabric. At this stage, a reinforcing member 325 can be connected to the leaflet attachment edge 118 of each leaflet 102a, 102b (as shown in FIG. 6A).

A third leaflet 102 (not shown in FIG. 8A; desirably already having a respective reinforcing member 325) can be similarly coupled to leaflets 102a, 102b by connecting a second connector 304 to the primary tab 106b of the leaflet 102a and to a primary tab of the third leaflet and connecting a third connector 304 to the primary tab 106a of the leaflet 102b and to the other primary tab of the third leaflet, thereby forming a leaflet assembly of three leaflets (similar to the assembly shown in FIG. 2) coupled to each other with respective connectors 304. It should be understood that the leaflet assembly can include additional leaflets coupled to each other with additional connectors 304.

The adjacent sub-commissure edges 124a, 124b (in FIGS. 3A-3B) of adjacent leaflets can be connected to each other with sutures 104, as shown in FIGS. 1 and 2. The sutures can, for example, form in-and-out stitches or whip stitches that extend through a pair of adjacent sub-commissure edges 124a, 124b.

The secondary tabs 108a, 108b of each leaflet 102 can then be folded downwardly against their corresponding primary tabs 106a, 106b. For example, referring to FIG. 8A, the secondary tab 108a of the leaflet 102a can be folded downwardly against the primary tab 106a of the leaflet 102a on the same side of the leaflet as the connector 304. In this manner, at least a portion of the secondary tab 108a can partially overlap a portion of the connector 304 (that is, a portion of the connector 304 is situated between the primary tab 106a and the second tab 108a). Similarly, the secondary tab 108b of the leaflet 102b can be folded downwardly against the primary tab 106b of the leaflet 102b.

Either before or after folding the secondary tabs 108a, 108b downwardly against the primary tabs 106a, 106b, the retention member 150 can be placed between the inner and outer fold layers 110a, 112a of the fold portion 107a and the inner and outer fold layers 110b, 112b of the fold portion 107b. The retention member 150 can secure the inner fold layers 110a, 110b together, such that the inner fold layers 110a, 110b are held against each other.

After folding the secondary tabs 108a, 108b, each of the secondary tabs 108a, 108b can be folded lengthwise along a vertical fold axis relative to the fold portions 107a, 107b to form an L-shape between the secondary tabs 108a, 108b and the fold portions 107a, 107b. The outer fold layers 112a, 112b can contact the inner surface of the leaflets and the secondary tabs 108a, 108b can contact the connector 304. The secondary tabs 108a, 108b can be sutured to the connector 304, such as with sutures 310 (shown in FIG. 8C).

Referring now to FIG. 8B, a commissure tab assembly formed by a connector 304, the tabs 106a, 108a of the leaflet 102a, and the tabs 106b, 108b of the leaflet 102b can be coupled to a commissure window 246 of a frame as follows. As shown in FIG. 8B, the connector 304 and the primary tabs 106a, 106b can be inserted through a commissure window 246 defined by a pair of struts 246a, 246b (from the inside of the frame to the outside of the frame), while the secondary tabs 108a, 108b remain inside the frame. The commissure tab assembly is then pressed inwardly at the wedge element 308 (in the direction of arrow 154) such that the primary tab 106a and a portion of the connector 304 are against the frame on one side of the window 246 and the primary tab 106b and a portion of the connector 304 are against the frame on the other side of the window 216.

As shown in FIG. 8C, the pressing of the commissure tab assembly also causes the primary tab 106a and a portion of the connector 304 to fold around the strut 216a on the outside of the frame opposite the secondary tab 108a, and the primary tab 106b and a portion of the connector 304 to fold around the strut 216b on the outside of the frame opposite the secondary tab 108b. A pair of suture lines 312 can be formed to retain the primary tabs 106a, 106b against the frame. Each suture line 312 extends through the connector 304, a primary tab, the wedge element 308, and another portion of the connector 304.

Each primary tab 106a, 106b can be secured to a corresponding secondary tab 108a, 108b with a primary suture line 314. Each suture line 314 extends through one layer of the connector 304, a primary tab 106a, 106b, another layer of the connector 304, another layer of the connector 304, and the secondary tab 108a, 108b. The end portions of the suture material used to form the primary suture lines 314 (or separate sutures) can be used to form whip stitches 316 at the adjacent outer edges of the tabs 106a, 108a and at the adjacent outer edges of the tabs 106b, 108b. A first set of stitches 316 can extend through the tabs 106a, 108a and two layers of the connector 304 between the tabs 106a, 108a, and a second set of stitches can extend through the tabs 106b, 108b and two layers of the connector 304 between the tabs 106b, 108b.

During valve cycling, the leaflets 102a, 102b can articulate primarily at inner edges 146a, 146b of the inner fold layers 110a, 110b. Specifically, the leaflets can articulate about a longitudinal axis 158 that is radially inward of the retention member 150. The riser portions 120a, 120b function to retain the retention members 150 slightly spaced above the coaptation portions 120c of the leaflets to avoid interference with the coaptation portions during working cycles of the prosthetic valve.

The remaining commissure tab assemblies of the leaflet assembly can be coupled to respective commissure windows 246 of the frame 200 in the same manner as described above. Further details of the method for forming the commissure tab assemblies and coupling them to the frame are disclosed in U.S. Pat. No. 9,393,110, which is incorporated herein by reference. It should be noted that FIGS. 8A-8C show one exemplary technique for coupling the commissures of a leaflet assembly to a frame. Other techniques, methods, and mechanisms can be used for coupling the commissures of the leaflet assembly to the frame 200, such as any of those disclosed in U.S. Pat. No. 9,393,110, U.S. Publication No. 2018/0325665, or U.S. Application No. 63/003,085, filed Mar. 31, 2020, which are incorporated herein by reference.

Further, it should be understood that the techniques and methods disclosed herein for forming a valvular structure and assembling it to the frame of a prosthetic valve can be applied to other types of prosthetic valves, such as balloon-expandable prosthetic valves (such as disclosed in U.S. Pat. Nos. 9,393,110 and 11,096,781, which are incorporated herein by reference) and self-expandable prosthetic valves (such as disclosed in U.S. Pat. No. 8,652,202, which is incorporated herein by reference). Moreover, the techniques and methods disclosed herein for forming a valvular structure and assembling it to the frame of a prosthetic valve can be applied to frames that have commissure windows for receiving commissures (such as disclosed in U.S. Pat. No. 9,393,110) or frames that do not have commissure windows (such as disclosed in U.S. Pat. Nos. 8,652,202 and 11,096,781).

Additionally, the retention members disclosed herein can be used to assemble a valvular structure having leaflets that have different configurations than shown in FIGS. 1-6 and 8A-8C. For example, leaflets can be similar to leaflets 102, but can have secondary tabs 108a, 108b extending directly from primary tabs 106a, 106b; that is, the secondary tabs 108a, 108b are not offset from the primary tabs 106a, 106b by fold portions 107a, 107b. An example of a leaflet having such a configuration is shown in U.S. Pat. No. 9,393,110. In such examples, when the leaflets are assembled with retention members (for example, retention members 150) to form a valvular structure, the secondary tabs are folded against the primary tabs and at each commissure, a retention member is positioned to extend between a primary tab and a secondary tab of a first leaflet and a primary tab and a secondary tab of a second leaflet. In other words, at each commissure, the primary tab and the secondary tab of the first leaflet form inner and outer fold layers, respectively, defining a first channel, and the primary tab and the secondary tab of the second leaflet form inner and outer fold layers, respectively, defining a second channel, wherein a respective retention member extends through the first and second channels.

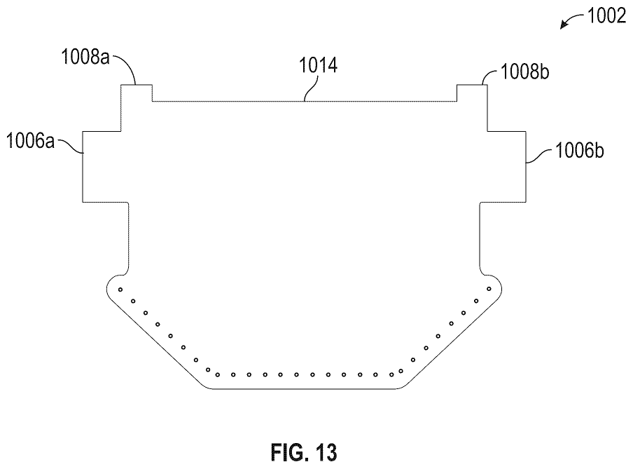

In some examples, the commissure portions of the leaflets need not include any fold portions that form inner and outer layers forming a channel to receive a retention member. Instead, at each commissure, a retention member (for example, retention member 150) can extend around a pair of adjacent commissure tabs of adjacent leaflets (without folding any portions of the leaflets to form a fold layer on the outside of the retention member). For example, as shown in FIG. 13, a leaflet 1002 can have tab portions 1008a, 1008b that extend axially from opposite ends of a free edge 1014 (similar to secondary tabs 108a, 108b, but which are not folded). A commissure can be formed by pairing first and second commissure tabs 1008a, 1008b of adjacent leaflets 1002 and positioning a retention member (for example, retention member 150) to extend around the first and second commissure tabs 1008a, 1008b. In the assembled state, the secondary tabs 1008a, 1008b extend axially beyond the free edge 1014 in the downstream direction.

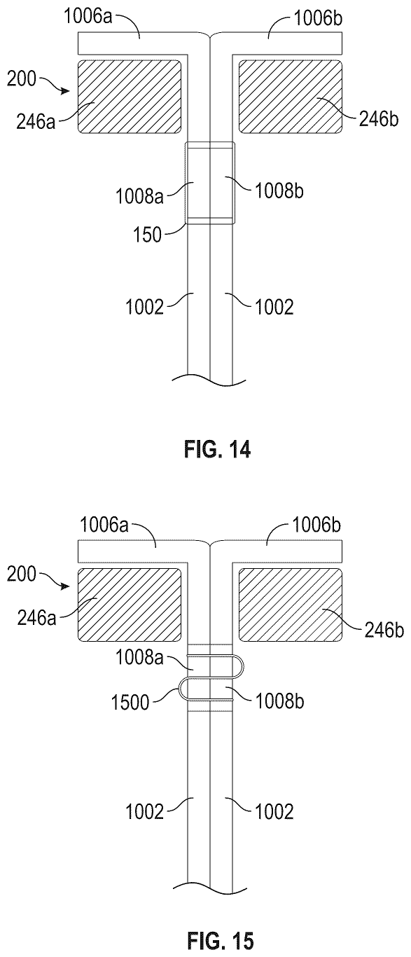

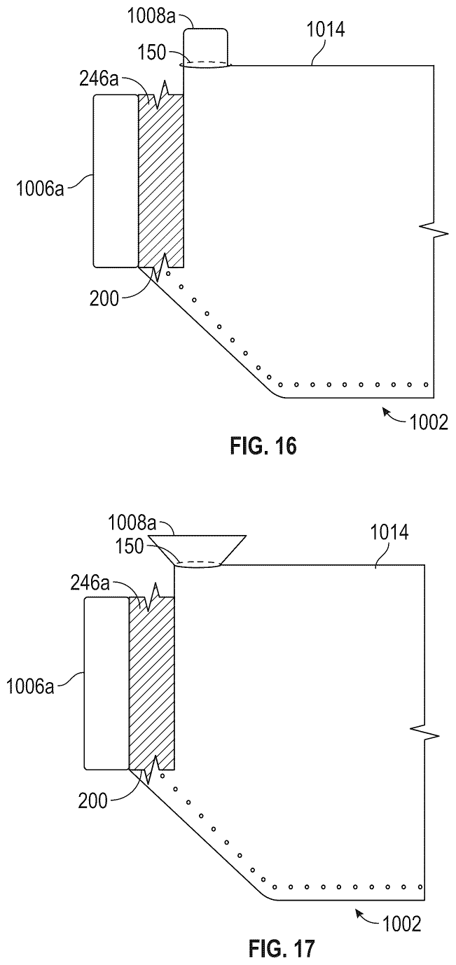

FIGS. 14-17 show examples of a commissure formed from two leaflets 1002 of the type shown in FIG. 13 in which a pair of primary tabs 1006a, 1006b of adjacent leaflets are coupled to struts 246a, 246b of a commissure window 246 of the frame 200. Specifically, FIGS. 14 and 15 show top views of a commissure formed by two adjacent leaflets 1002 without folding the tab portions 1008a, 1008b. As shown in FIGS. 14 and 16, a retention member 150 can extend around the tab portions 1008a, 1008b of the two adjacent leaflets 1002 to hold the adjacent tab portions 1008a, 1008b together during working cycles of a prosthetic valve (for example, prosthetic valve 300 of FIG. 6A). The primary tabs 1006a, 1006b can be coupled to the struts 246a, 246b using any of various known techniques or mechanisms, such as with sutures extending through the primary tabs and around the struts.

In some examples, the tabs portions 1008a, 1008b of the two adjacent leaflets 1002 can be coupled together with a retention member in the form of stitches 1500 (for example, formed with a suture), as shown in FIG. 15. As shown, the stitches 1500 can extend through the tab portions 1008a, 1008b in an in-and-out pattern extending in a radial direction along the tab portions. In other examples, the stitches 1500 can be whip stitches that extend around the upper edges of the tab portion 1008a, 1008b and through the tab portions, forming a pattern of whip stitches extending in a radial direction. The stitches 1500 can be in addition to the retention member 150 or an alternative to the retention member 150. As such, in some examples, the stitches 1500 can also be referred to as a retention member.

In some examples, the tab portions 1008a, 1008b can have a square or rectangular shape (FIG. 16). In other examples, each tab portion 1008a, 1008b can be wider at a location that is axially above the location where the retention member 150 is coupled to the tab portion 1008a (adjacent to the free edge 1014). The greater width of the tab portions 1008a, 1008b at a location axially above the retention member 150 can help prevent axial movement of the retention member 150 relative to the tab portions 1008a, 1008b. For example, as shown in FIG. 17, the tab portion 1008a can have a trapezoid shape having a greater width at the top or free edge of the tab portion 1008a than at the bottom of the tab portion 1008a where it is connected to the free edge 1014. While illustrated as a trapezoid in FIG. 17, in other examples, the tab portions 1008a, 1008b can comprise other shapes having a width that is greater at a location axially above the retention member 150.

As introduced above, the threaded rods 226 can removably couple the prosthetic valve 300 to actuator assemblies of a delivery apparatus. Referring to FIG. 9, it illustrates an example of a delivery apparatus 400 for delivering a prosthetic device or valve 402 (for example, prosthetic valve 300) to a desired implantation location. The prosthetic valve 402 can be releasably coupled to the delivery apparatus 400. It should be understood that the delivery apparatus 400 and other delivery apparatuses disclosed herein can be used to implant prosthetic devices other than prosthetic valves, such as stents or grafts.

The delivery apparatus 400 in the illustrated example generally includes a handle 404, a first elongated shaft 406 (which comprises an outer shaft in the illustrated example) extending distally from the handle 404, at least one actuator assembly 408 extending distally through the first shaft 406, a second elongated shaft 409 (which comprises an inner shaft in the illustrated example) extending through the first shaft 406, and a nosecone 410 coupled to a distal end portion of the second shaft 409. The second shaft 409 and the nosecone 410 can define a guidewire lumen for advancing the delivery apparatus through a patient's vasculature over a guidewire. The at least one actuator assembly 408 can be configured to radially expand and/or radially collapse the prosthetic valve 402 when actuated, such as by one or more knobs 411, 412, 414 included on the handle 404 of the delivery apparatus 400.

Though the illustrated example shows two actuator assemblies 408 for purposes of illustration, it should be understood that one actuator assembly 408 can be provided for each actuator (for example, actuator or threaded rod 226) on the prosthetic valve. For example, three actuator assemblies 408 can be provided for a prosthetic valve having three actuators. In other examples, a greater or fewer number of actuator assemblies can be present.

In some examples, a distal end portion 416 of the shaft 406 can be sized to house the prosthetic valve in its radially compressed, delivery state during delivery of the prosthetic valve through the patient's vasculature. In this manner, the distal end portion 416 functions as a delivery sheath or capsule for the prosthetic valve during delivery,

The actuator assemblies 408 can be releasably coupled to the prosthetic valve 402. For example, in the illustrated example, each actuator assembly 408 can be coupled to a respective actuator (for example, threaded rod 226) of the prosthetic valve 402. Each actuator assembly 408 can comprise a support tube and an actuator member. When actuated, the actuator assembly can transmit pushing and/or pulling forces to portions of the prosthetic valve to radially expand and collapse the prosthetic valve as previously described. The actuator assemblies 408 can be at least partially disposed radially within, and extend axially through, one or more lumens of the first shaft 406. For example, the actuator assemblies 408 can extend through a central lumen of the shaft 406 or through separate respective lumens formed in the shaft 406.

The handle 404 of the delivery apparatus 400 can include one or more control mechanisms (for example, knobs or other actuating mechanisms) for controlling different components of the delivery apparatus 400 in order to expand and/or deploy the prosthetic valve 402. For example, in the illustrated example the handle 404 comprises first, second, and third knobs 411, 412, and 414, respectively.

The first knob 411 can be a rotatable knob configured to produce axial movement of the first shaft 406 relative to the prosthetic valve 402 in the distal and/or proximal directions in order to deploy the prosthetic valve from the delivery sheath 416 once the prosthetic valve has been advanced to a location at or adjacent the desired implantation location with the patient's body. For example, rotation of the first knob 411 in a first direction (for example, clockwise) can retract the sheath 416 proximally relative to the prosthetic valve 402 and rotation of the first knob 411 in a second direction (for example, counter-clockwise) can advance the sheath 416 distally. In other examples, the first knob 411 can be actuated by sliding or moving the first knob 411 axially, such as pulling and/or pushing the knob. In other examples, actuation of the first knob 411 (rotation or sliding movement of the first knob 411) can produce axial movement of the actuator assemblies 408 (and therefore the prosthetic valve 402) relative to the delivery sheath 416 to advance the prosthetic valve distally from the sheath 416.

The second knob 412 can be a rotatable knob configured to produce radial expansion and/or compression of the prosthetic valve 402. For example, rotation of the second knob 412 can rotate the threaded rods of the prosthetic valve 402 via the actuator assemblies 408. Rotation of the second knob 412 in a first direction (for example, clockwise) can radially expand the prosthetic valve 402 and rotation of the second knob 412 in a second direction (for example, counter-clockwise) can radially collapse the prosthetic valve 402. In other examples, the second knob 412 can be actuated by sliding or moving the second knob 412 axially, such as pulling and/or pushing the knob.

The third knob 414 can be a rotatable knob operatively connected to a proximal end portion of each actuator assembly 408. The third knob 414 can be configured to retract an outer sleeve or support tube of each actuator assembly 408 to disconnect the actuator assemblies 408 from the proximal portions of the actuators of the prosthetic valve (for example, threaded rod). Once the actuator assemblies 408 are uncoupled from the prosthetic valve 402, the delivery apparatus 400 can be removed from the patient, leaving just the prosthetic valve 402 in the patient.

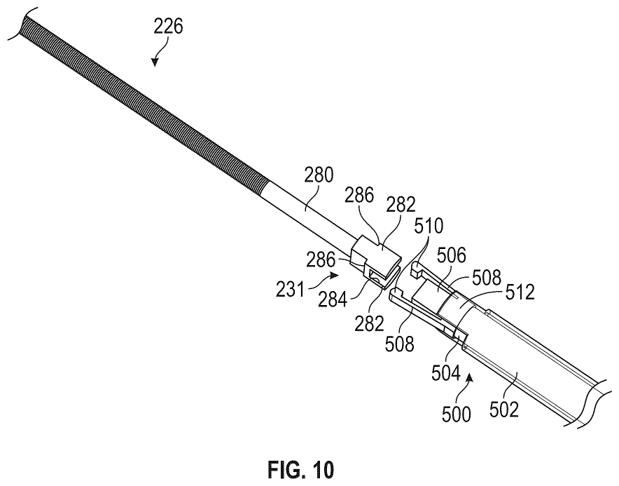

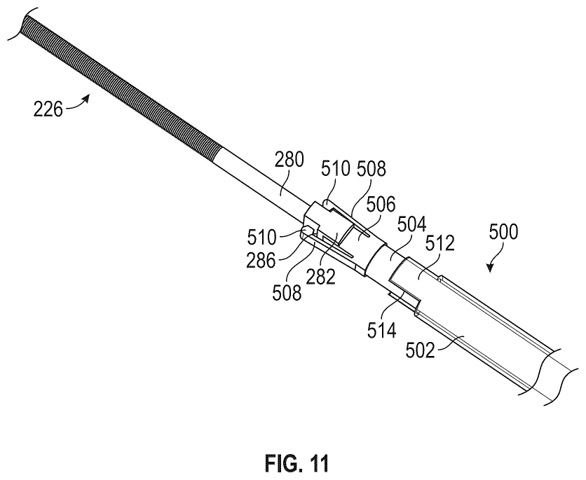

Referring to FIGS. 10-11, they illustrate how each of the threaded rods 226 of the prosthetic device 300 can be removably coupled to an actuator assembly 500 (for example, actuator assemblies 408) of a delivery apparatus (for example, delivery apparatus 400), according to one example. Specifically, FIG. 11 illustrates how one of the threaded rods 226 can be coupled to an actuator assembly 500, while FIG. 10 illustrates how the threaded rod 226 can be detached from the actuator assembly 500.

As introduced above, an actuator assembly 500 can be coupled to the head portion 231 of each threaded rod 226. The head portion 231 can be included at a proximal end portion 180 of the threaded rod 226 and can extend proximally past a proximal end of the second post 224 (FIG. 7A). The head portion 231 can comprise first and second protrusions 282 defining a channel or slot 284 between them, and one or more shoulders 286. As discussed above, the head portion 231 can have a width greater than a diameter of the inner bore of the second post 224 such that the head portion 231 is prevented from moving into the inner bore of the second post 224 and such that the head portion 231 abuts the outflow end 208 of the frame 200. In particular, the head portion 231 can abut an outflow apex 219b of the frame 200. The head portion 231 can be used to apply a distally-directed force to the second post 224, for example, during radial expansion of the frame 200.

Each actuator assembly 500 can comprise a first actuation member configured as a support tube or outer sleeve 502 and a second actuation member configured as a driver 504. The driver 504 can extend through the outer sleeve 502. The outer sleeve 502 is shown transparently in FIGS. 10-11 for purposes of illustration. The distal end portions of the outer sleeve 502 and driver 504 can be configured to engage or abut the proximal end of the threaded rod 226 (for example, the head portion 231) and/or the frame 200 (for example, the apex 219b). The proximal portions of the outer sleeve 502 and driver 504 can be operatively coupled to the handle of a delivery apparatus (for example, handle 404). The delivery apparatus in this example can include the same features described previously for delivery apparatus 400. In particular examples, the proximal end portions of each driver 504 can be operatively connected to the knob 412 such that rotation of the knob 412 (clockwise or counterclockwise) causes corresponding rotation of the drivers 504. The proximal end portions of each outer sleeve 502 can be operatively connected to the knob 414 such that rotation of the knob 414 (clockwise or counterclockwise) causes corresponding axial movement of the sleeves 502 (proximally or distally) relative to the drivers 504. In other examples, the handle can include electric motors for actuating these components.

The distal end portion of the driver 504 can comprise a central protrusion 506 configured to extend into the slot 284 of the threaded rod 226, and one or more flexible elongated elements or arms 508 including protrusions or teeth 510 configured to be releasably coupled to the shoulders 286 of the threaded rod 226. The protrusions 510 can extend radially inwardly toward a longitudinal axis of the second actuation member 504. As shown in FIGS. 10-11, the elongated elements 508 can be configured to be biased radially outward to an expanded state, for example, by shape setting the elements 508.

As shown in FIG. 11, to couple the actuator assembly 500 to the threaded rod 226, the driver 504 can be positioned such that the central protrusion 506 is disposed within the slot 184 (FIG. 10) and such that the protrusions 510 of the elongated elements 508 are positioned distally to the shoulders 286. As the outer sleeve 502 is advanced (for example, distally) over the driver 504, the sleeve 502 compresses the elongated elements 508 they abut and/or snap over the shoulders 286, thereby coupling the actuator assembly 500 to the threaded rod 226. Thus, the outer sleeve 502 effectively squeezes and locks the elongated elements 508 and the protrusions 510 of the driver 504 into engagement with (that is, over) the shoulders 286 of the threaded rod 226, thereby coupling the driver 504 to the threaded rod 226.

Because the central protrusion 506 of the driver 504 extends into the slot 284 of the threaded rod 226 when the driver 504 and the threaded rod 226 are coupled, the driver 504 and the threaded rod 226 can be rotational locked such that they co-rotate. So coupled, the driver 504 can be rotated (for example, using knob 412 the handle of the delivery apparatus 400) to cause corresponding rotation of the threaded rod 226 to radially expand or radially compress the prosthetic device. The central protrusion 506 can be configured (for example, sized and shaped) such that it is advantageously spaced apart from the inner walls of the outer sleeve 502, such that the central protrusion 506 does not frictionally contact the outer sleeve 502 during rotation. Though in the illustrated example the central protrusion 506 has a substantially rectangular shape in cross-section, in other examples, the protrusion 506 can have any of various shapes, for example, square, triangular, oval, etc. The slot 284 can be correspondingly shaped to receive the protrusion 506.

The outer sleeve 502 can be advanced distally relative to the driver 504 past the elongated elements 508, until the outer sleeve 502 engages the frame 200 (for example, a second post 224 of the frame 200). The distal end portion of the outer sleeve 502 also can comprise first and second support extensions 512 defining gaps or notches 514 between the extensions 512. The support extensions 512 can be oriented such that, when the actuator assembly 500 is coupled to a respective threaded rod 226, the support extensions 512 extend partially over an adjacent end portion (for example, the upper end portion) of one of the second posts 224 on opposite sides of the post 224. The engagement of the support extensions 512 with the frame 200 in this manner can counter-act rotational forces applied to the frame 200 by the rods 226 during expansion of the frame 200. In the absence of a counter-force acting against these rotational forces, the frame can tend to “jerk” or rock in the direction of rotation of the rods when they are actuated to expand the frame. The illustrated configuration is advantageous in that outer sleeves, when engaging the proximal posts 224 of the frame 200, can prevent or mitigate such jerking or rocking motion of the frame 200 when the frame 200 is radially expanded.

To decouple the actuator assembly 500 from the prosthetic device 300, the sleeve 502 can be withdrawn proximally relative to the driver 504 until the sleeve 502 no longer covers the elongated elements 508 of the driver 504. As described above, the sleeve 502 can be used to hold the elongated elements 508 against the shoulders 286 of the threaded rod 226 since the elongated elements 508 can be naturally biased to a radial outward position where the elongated elements 508 do not engage the shoulders 186 of the threaded rod 226. Thus, when the sleeve 502 is withdrawn such that it no longer covers/constrains the elongated elements 508, the elongated elements 508 can naturally and/or passively deflect away from, and thereby release from, the shoulders 286 of the threaded rod 226, thereby decoupling the driver 504 from the threaded rod 226.

The sleeve 502 can be advanced (moved distally) and/or retracted (moved proximally) relative to the driver 504 via a control mechanism (for example, knob 414) on the handle 404 of the delivery apparatus 400, by an electric motor, and/or by another suitable actuation mechanism. For example, the physician can turn the knob 414 in a first direction to apply a distally directed force to the sleeve 502 and can turn the knob 414 in an opposite second direction to apply a proximally directed force to the sleeve 502. Thus, when the sleeve 502 does not abut the prosthetic device and the physician rotates the knob 414 in the first direction, the sleeve 502 can move distally relative to the driver 504, thereby advancing the sleeve 502 over the driver 504. When the sleeve 502 does abut the prosthetic device, the physician can rotate the knob 414 in the first direction to push the entire prosthetic device distally via the sleeve 502. Further, when the physician rotates the knob 414 in the second direction the sleeve 502 can move proximally relative to the driver 504, thereby withdrawing/retracting the sleeve 502 from the driver 504.

Once the prosthetic valve 300 is implanted, the leaflets of the valve can cycle between closed and open states during the diastolic and systolic phases. When the retention members 150 are used to securely hold the commissure portions of the leaflets, the leaflets are prevented from splaying apart at the commissure portions, such that leaflet fluttering and/or leaflet contact with the frame is reduced which improves the durability of the leaflets. Examples A-B below compare the leaflets with retention members as in FIG. 4A with an alternative leaflet design.

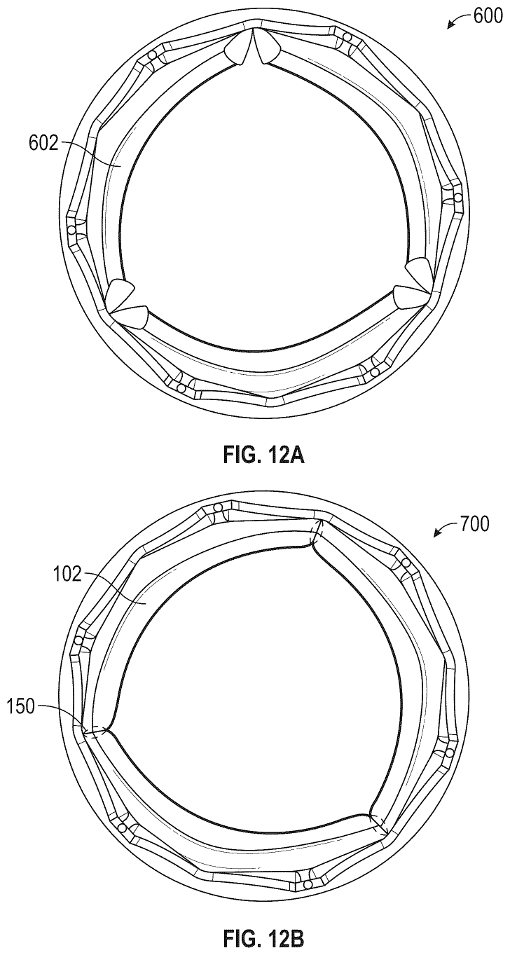

EXAMPLE A (COMPARATIVE): A prosthetic valve 600 was constructed using three leaflets 602 with a valve diameter of 30 mm. FIG. 12A shows an illustration of a frame of a video recording made as the leaflets transitioned between open and closed states. As shown, the prosthetic valve 600 does not include the retention members 150. In the frame illustrated in FIG. 12A, the leaflets 602 are in an open state. As shown, the leaflets 602 separate or splay apart at each commissure, which may result in increased leaflet fluttering and/or leaflet contact with the frame.

EXAMPLE B: A prosthetic valve 700 was constructed using the leaflets 102 and the retention members 150 with a valve diameter of 30 mm. FIG. 12B shows an illustration of a frame of a video recording made as the leaflets transitioned between open and closed states. In the video frame illustrated in FIG. 12B, the leaflets are in an open state. As shown in FIG. 12B, the retention members 150 prevent the leaflets 102 from separating at the commissures, which may result in decreased leaflet fluttering and/or leaflet contact with the frame.

### Delivery Techniques

For implanting a prosthetic valve within the native aortic valve via a transfemoral delivery approach, the prosthetic valve is mounted in a radially compressed state along the distal end portion of a delivery apparatus. The prosthetic valve and the distal end portion of the delivery apparatus are inserted into a femoral artery and are advanced into and through the descending aorta, around the aortic arch, and through the ascending aorta. The prosthetic valve is positioned within the native aortic valve and radially expanded (for example, by inflating a balloon, actuating one or more actuators of the delivery apparatus, or deploying the prosthetic valve from a sheath to allow the prosthetic valve to self-expand). Alternatively, a prosthetic valve can be implanted within the native aortic valve in a transapical procedure, whereby the prosthetic valve (on the distal end portion of the delivery apparatus) is introduced into the left ventricle through a surgical opening in the chest and the apex of the heart and the prosthetic valve is positioned within the native aortic valve. Alternatively, in a transaortic procedure, a prosthetic valve (on the distal end portion of the delivery apparatus) are introduced into the aorta through a surgical incision in the ascending aorta, such as through a partial J-sternotomy or right parasternal mini-thoracotomy, and then advanced through the ascending aorta toward the native aortic valve.

For implanting a prosthetic valve within the native mitral valve via a transseptal delivery approach, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, into the right atrium, across the atrial septum (through a puncture made in the atrial septum), into the left atrium, and toward the native mitral valve. Alternatively, a prosthetic valve can be implanted within the native mitral valve in a transapical procedure, whereby the prosthetic valve (on the distal end portion of the delivery apparatus) is introduced into the left ventricle through a surgical opening in the chest and the apex of the heart and the prosthetic valve is positioned within the native mitral valve.

For implanting a prosthetic valve within the native tricuspid valve, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, and into the right atrium, and the prosthetic valve is positioned within the native tricuspid valve. A similar approach can be used for implanting the prosthetic valve within the native pulmonary valve or the pulmonary artery, except that the prosthetic valve is advanced through the native tricuspid valve into the right ventricle and toward the pulmonary valve/pulmonary artery.

Another delivery approach is a transatrial approach whereby a prosthetic valve (on the distal end portion of the delivery apparatus) is inserted through an incision in the chest and an incision made through an atrial wall (of the right or left atrium) for accessing any of the native heart valves. Atrial delivery can also be made intravascularly, such as from a pulmonary vein. Still another delivery approach is a transventricular approach whereby a prosthetic valve (on the distal end portion of the delivery apparatus) is inserted through an incision in the chest and an incision made through the wall of the right ventricle (typically at or near the base of the heart) for implanting the prosthetic valve within the native tricuspid valve, the native pulmonary valve, or the pulmonary artery.

In all delivery approaches, the delivery apparatus can be advanced over a guidewire previously inserted into a patient's vasculature. Moreover, the disclosed delivery approaches are not intended to be limited. Any of the prosthetic valves disclosed herein can be implanted using any of various delivery procedures and delivery devices known in the art.

Any of the systems, devices, apparatuses, etc. herein can be sterilized (for example, with heat, radiation, and/or chemicals, etc.) to ensure they are safe for use with patients, and any of the methods herein can include sterilization of the associated system, device, apparatus, etc. as one of the steps of the method. Examples of radiation for use in sterilization include, without limitation, gamma radiation and ultra-violet radiation. Examples of chemicals for use in sterilization include, without limitation, ethylene oxide and hydrogen peroxide.

### Additional Examples of the Disclosed Technology

In view of the above-described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A prosthetic valve comprising: an annular frame; a plurality of leaflets positioned within and coupled to the annular frame, each leaflet comprising a body portion having a free outflow edge and two opposing commissure portions on opposite sides of the leaflet, wherein each commissure portion is folded along a radially-extending fold line to form an inner fold layer and an outer fold layer, wherein each commissure portion is paired with a commissure portion of an adjacent leaflet to form a commissure that is coupled to the frame; and for each commissure, a retention member extending between the inner fold layer and the outer fold layer of a first commissure portion of the commissure and between the inner fold layer and the outer fold layer of a second commissure portion of the commissure to hold the first and second commissure portions together during working cycles of the prosthetic valve.

Example 2. The prosthetic valve of any example herein, particularly example 1, wherein the retention member comprises a loop.

Example 3. The prosthetic valve of any example herein, particularly either example 1 or example 2, wherein the retention member comprises an elongate member having two ends that are tied together to form the loop.

Example 4. The prosthetic valve of any example herein, particularly any one of examples 1-3, wherein the retention member comprises a suture.

Example 5. The prosthetic valve of any example herein, particularly either example 1 or example 2, wherein the retention member is a pre-formed, continuous band.

Example 6. The prosthetic valve of any example herein, particularly any one of examples 1-5, wherein the retention member is not sutured to the leaflets.

Example 7. The prosthetic valve of any example herein, particularly any one of examples 1-6, wherein the retention member does not pierce any portions of the leaflets.

Example 8. The prosthetic valve of any example herein, particularly any one of examples 1-7, wherein the leaflets articulate at a location radially inward of the retention member.

Example 9. The prosthetic valve of any example herein, particularly any one of examples 1-8, wherein the free outflow edge includes a coaptation portion and two step portions, wherein the step portions axially offset the retention member from the coaptation portion.

Example 10. The prosthetic valve of any example herein, particularly any one of examples 1-9, wherein the commissure portions of each leaflet comprise two opposing primary tabs extending from opposite sides of the body portion and two opposing secondary tabs, wherein the primary tabs extend from the inner fold layers and the secondary tabs extend from the outer fold layers.

Example 11. The prosthetic valve of any example herein, particularly any one of examples 1-10, wherein the retention member extends continuously in a closed loop through the inner and outer fold layers of the first commissure portion, around radial inner edges of the inner fold layers of the first and second commissure portions, through the inner and outer fold layers of the second commissure portion and around radial outer edges of the inner fold layers of the first and second commissure portions.

Example 12. The prosthetic valve of a any example herein, particularly ny of examples 1-11, wherein the annular frame comprises a plurality of commissure posts connected to the commissures.

Example 13. The prosthetic valve of any example herein, particularly example 12, wherein the commissure posts comprise openings through which portions of the commissures extend.

Example 14. The prosthetic valve of any example herein, particularly any of examples 1-13, wherein the retention member extends radially inwardly from the annular frame.

Example 15. The prosthetic valve of any example herein, particularly any of examples 1-14, wherein the retention member is oriented in a horizontal plane that is perpendicular to a longitudinal axis of the annular frame.

Example 16. A prosthetic valve comprising: an annular frame; and a valvular structure mounted within the annular frame, the valvular structure comprising a plurality of leaflets having commissure portions that define a plurality of commissures and a plurality of retention members, wherein each retention member extends through two radially-extending channels defined by commissure portions of two adjacent leaflets and is configured to hold the commissure portions of the two adjacent leaflets together during working cycles of the valvular structure.

Example 17. The prosthetic valve of any example herein, particularly example 16, wherein each retention member comprises a loop.

Example 18. The prosthetic valve of any example herein, particularly either example 16 or example 17, wherein each retention member comprises an elongate member having two ends that are tied together to form the loop.

Example 19. The prosthetic valve of any example herein, particularly any one of examples 16-18, wherein each retention member comprises a suture that does not pierce any portions of the leaflets.

Example 20. The prosthetic valve of any example herein, particularly either example 16 or example 17, wherein each retention member is a pre-formed, continuous band.

Example 21. The prosthetic valve of any example herein, particularly any one of examples 16-20, wherein none of the retention members are sutured to the leaflets.

Example 22. The prosthetic valve of any example herein, particularly any one of examples 16-21, wherein the leaflets articulate at a location radially inward of each retention member.

Example 23. The prosthetic valve of any example herein, particularly any one of examples 16-22, wherein each leaflet includes a free outflow edge having a coaptation portion and two step portions, wherein the step portions axially offset the retention member from the coaptation portion.

Example 24. The prosthetic valve of any example herein, particularly any of examples 16-23, wherein the channel of each commissure portion is defined by an inner fold layer and an outer fold layer of the commissure portion.

Example 25. The prosthetic valve of any example herein, particularly example 24, wherein each commissure portion comprises a primary tab extending from the inner fold layer and a secondary tab extending from the outer fold layer.

Example 26. The prosthetic valve of any example herein, particularly either example 24 or example 25, wherein the retention member extends continuously in a closed loop through inner and outer fold layers of a first commissure portion, around radial inner edges of the inner fold layers of the first commissure portion and radial inner edges of inner fold layers of a second commissure portion, through the inner fold layers and outer fold layers of the second commissure portion and around radial outer edges of the inner fold layers of the first and second commissure portions.

Example 27. The prosthetic valve of any example herein, particularly any of examples 16-26, wherein each retention member extends radially inwardly from the annular frame.

Example 28. The prosthetic valve of any example herein, particularly any of examples 16-27, wherein each retention member is oriented in a horizontal plane that is perpendicular to a longitudinal axis of the annular frame.

Example 29. A prosthetic valve comprising: an annular frame; a plurality of leaflets positioned within and coupled to the annular frame, each leaflet comprising a free outflow edge and two opposing commissure portions on opposite sides of the leaflet, wherein each commissure portion is paired with a commissure portion of an adjacent leaflet to form a commissure that is coupled to the frame; and for each commissure, a retention member coupled to two adjacent commissure portions to hold the adjacent commissure portions together during working cycles of the prosthetic valve, wherein the retention member extends in a radial inward direction relative to the annular frame.

Example 30. The prosthetic valve of any example herein, particularly example 29, wherein the retention member comprises a loop.

Example 31. The prosthetic valve of any example herein, particularly either example 29 or example 30, wherein the retention member comprises an elongate member having two ends that are tied together to form the loop.

Example 32. The prosthetic valve of any example herein, particularly any one of examples 29-31, wherein the retention member comprises a suture.

Example 33. The prosthetic valve of any example herein, particularly either example 29 or example 30, wherein the retention member is a pre-formed, continuous band.

Example 34. The prosthetic valve of any example herein, particularly any one of examples 29-33, wherein the retention member is not sutured to the leaflets.

Example 35. The prosthetic valve of any example herein, particularly any one of examples 29-34, wherein the leaflets articulate at a location radially inward of the retention member.

Example 36. The prosthetic valve of any example herein, particularly any one of examples 29-35, wherein each free outflow edge includes a coaptation portion and two step portions, wherein the step portions axially offset the retention member from the coaptation portion.

Example 37. The prosthetic valve of any example herein, particularly any one of examples 29-36, wherein each commissure portion of each leaflet comprises two opposing primary tabs extending from opposite sides of the leaflet and two opposing secondary tabs, wherein the secondary tabs are folded against the primary tabs such that the primary tabs form inner fold layers and the secondary tabs form outer fold layers.

Example 38. The prosthetic valve of any example herein, particularly example 37, wherein the retention member extends between an inner fold layer and an outer fold layer of a first commissure portion and between an inner fold layer and an outer fold layer of a second commissure portion to hold the first and second commissure portions together during a working cycle of the prosthetic valve.

Example 39. The prosthetic valve of any example herein, particularly example 38, wherein the retention member extends continuously in a closed loop through the inner and outer fold layers of the first commissure portion, around radial inner edges of the inner fold layers of the first and second commissure portions, through the inner and outer fold layers of the second commissure portion and around radial outer edges of the inner fold layers of the first and second commissure portions.

Example 40. The prosthetic valve of any example herein, particularly any one of examples 29-36, wherein each commissure portion of each leaflet comprises two opposing primary tabs extending from opposite sides of the leaflet and two opposing secondary tabs disposed at opposite sides of the free outflow edge, wherein the retention member is coupled to a pair of adjacent secondary tabs of adjacent leaflets.

Example 41. The prosthetic valve of any example herein, particularly example 40, wherein the secondary tabs are not folded.

Example 42. The prosthetic valve of any example herein, particularly either example 40 or example 41, wherein each secondary tab extends axially from the free outflow edge, wherein a width of a secondary tab is greater at a location axially away from the retention member than a location where the retention member is coupled to the secondary tab.

Example 43. The prosthetic valve of any example herein, particularly any one of examples 29-42, wherein the retention member is disposed in a horizontal plane that is perpendicular to a longitudinal axis of the annular frame.

Example 44. The prosthetic valve of any example herein, particularly example 29, wherein each commissure portion of each leaflet comprises two opposing primary tabs extending from opposite sides of the leaflet and two opposing secondary tabs disposed at opposite sides of the free outflow edge, wherein the retention member comprises stitches extending through a pair of adjacent secondary tabs of adjacent leaflets.

Example 45. A delivery apparatus comprising: a delivery device comprising a handle; and a prosthetic valve releasably coupled to the delivery device, the prosthetic valve comprising an annular frame and a valvular structure mounted within the annular frame, the valvular structure comprising a plurality of leaflets having fold portions that define a plurality of commissures and a plurality of retention members, wherein each retention member extends through two radially-extending channels defined by fold portions of two adjacent leaflets and is configured to hold the fold portions of the two adjacent leaflets together during working cycles of the valvular structure.

Example 46. The delivery apparatus of any example herein, particularly example 45, wherein each retention member comprises a loop.

Example 47. The delivery apparatus of any example herein, particularly either example 45 or example 46, wherein each retention member comprises an elongate member having two ends that are tied together to form the loop.

Example 48. The delivery apparatus of any example herein, particularly any one of examples 45-47, wherein each retention member comprises a suture.

Example 49. The delivery apparatus of any example herein, particularly either example 45 or example 46, wherein each retention member is a pre-formed, continuous band.

Example 50. The delivery apparatus of any example herein, particularly any one of examples 45-49, wherein none of the retention members are sutured to the leaflets.

Example 51. The delivery apparatus of any example herein, particularly any one of examples 45-50, wherein the leaflets articulate at a location radially inward of each retention member.

Example 52. The delivery apparatus of any example herein, particularly any one of examples 45-51, wherein each leaflet includes a free outflow edge having a coaptation portion and two step portions, wherein the step portions axially offset the retention member from the coaptation portion.

Example 53. The delivery apparatus of any example herein, particularly any one of examples 45-52, wherein two opposing sides of each leaflet comprise a primary tab extending from a side of the leaflet and a secondary tab extending from a fold portion of the leaflet, wherein the fold portion is folded to align the primary tab and the secondary tab, and wherein each fold portion forms an inner fold layer and an outer fold layer.

Example 54. The delivery apparatus of any example herein, particularly example 53, wherein the channel is defined by the inner fold layers and the outer fold layers of the fold portions.

Example 55. The delivery apparatus of any example herein, particularly either example 53 or example 54, wherein a retention member extends continuously in a closed loop through inner and outer fold layers of a first leaflet, around radial inner edges of the inner fold layers of the first leaflet and radial inner edges of inner fold layers of a second leaflet, through the inner fold layers and outer fold layers of the second leaflet and around radial outer edges of the inner fold layers of the first and second leaflets.

Example 56. A method of assembling a prosthetic valve, the method comprising: forming a valvular structure that includes a plurality of leaflets that define a plurality of commissures and a radially-extending retention member at each commissure; and coupling the valvular structure to a frame.

Example 57. The method of any example herein, particularly example 56, wherein forming the valvular structure comprises: for each leaflet of the plurality of leaflets, folding a commissure portion of the leaflet along a radially extending fold line to form an inner fold layer and an outer fold layer; and positioning the retention member for each commissure between the inner fold layer and the outer fold layer of a first commissure portion of the commissure and between the inner fold layer and the outer fold layer of a second commissure portion of the commissure to hold the first and second commissure portions together during working cycles of the prosthetic valve.

Example 58. The method of any example herein, particularly either example 56 or example 57, wherein coupling the valvular structure to the frame comprises: positioning a portion of each commissure through an opening of a commissure post of the frame; and securing the portion of each commissure to the frame.

Example 59. A prosthetic valve comprising: an annular frame; a plurality of leaflets positioned within and coupled to the annular frame, each leaflet comprising a free outflow edge, two opposing primary tabs extending from opposite sides of the leaflet and two opposing secondary tabs disposed at opposite sides of the free outflow edge and extending axially beyond the free edge in downstream direction, wherein each primary tab is paired with a primary of an adjacent leaflet to form a commissure that is coupled to the frame; and at each commissure, a retention member connecting a pair of adjacent secondary tabs of adjacent leaflets to hold the secondary tabs together during working cycles of the prosthetic valve.

Example 60. The prosthetic valve of any example herein, particularly example 59, wherein each secondary tab has a constant width from a lower edge to an upper edge of the secondary tab.

Example 61. The prosthetic valve of any example herein, particularly example 59, wherein each secondary tab has a width that increases from a lower edge to an upper edge of the secondary tab.

Example 62. The prosthetic valve of any example herein, particularly example 61, wherein each secondary tab has a trapezoidal shape.

Example 63. The prosthetic valve of any example herein, particularly any one of examples 59-62, wherein the leaflets articulate at a location radially inward of the retention member.

Example 64. The prosthetic valve of any example herein, particularly any of examples 59-63, wherein the retention member comprises stitches that extend through a corresponding pair of secondary tabs.

Example 65. The prosthetic valve of any example herein, particularly any of one of examples 59-63, wherein the retention member comprises a loop.

Example 66. The prosthetic valve of any example herein, particularly example 65, wherein the retention member comprises an elongate member having two ends that are tied together to form the loop.

Example 67. The prosthetic valve of any example herein, particularly any one of examples 65-66, wherein the retention member comprises a suture.

Example 68. The prosthetic valve of any example herein, particularly example 65, wherein the retention member is a pre-formed, continuous band.

Example 69. The prosthetic valve of any example herein, particularly any one of examples 65-68, wherein the retention member is not sutured to the leaflets.

Example 70. The prosthetic valve of any example herein, particularly any one of examples 59-69, wherein at each commissure, the primary tabs are coupled to a pair of struts of the frame.

Example 71. The prosthetic valve of any example herein, particularly example 70, wherein at each commissure, the primary tabs extend through an opening defined between the pair of struts.

Example 72. The delivery apparatus or prosthetic valve of any example herein, particularly any of examples 1-71, wherein the delivery apparatus or prosthetic valve is sterilized.

The features described herein with regard to any example can be combined with other features described in any one or more of the other examples, unless otherwise stated. For example, any one or more features of one delivery apparatus can be combined with any one or more features of another delivery apparatus.

In view of the many possible ways in which the principles of the disclosure may be applied, it should be recognized that the illustrated configurations depict examples of the disclosed technology and should not be taken as limiting the scope of the disclosure nor the claims. Rather, the scope of the claimed subject matter is defined by the following claims and their equivalents.

In view of the many possible ways in which the principles of the disclosure may be applied, it should be recognized that the illustrated configurations depict examples of the disclosed technology and should not be taken as limiting the scope of the disclosure nor the claims. Rather, the scope of the claimed subject matter is defined by the following claims and their equivalents.

We claim:

1. A prosthetic valve comprising:
an annular frame;
a plurality of leaflets positioned within and coupled to the annular frame, each leaflet comprising a body portion having a free outflow edge and two opposing commissure portions on opposite sides of the leaflet, wherein each commissure portion is folded along a radially-extending fold line to form an inner fold layer and an outer fold layer, wherein each commissure portion is paired with a commissure portion of an adjacent leaflet to form a commissure that is coupled to the frame; and
for each commissure, a retention member extending between the inner fold layer and the outer fold layer of a first commissure portion of the commissure and between the inner fold layer and the outer fold layer of a second commissure portion of the commissure to hold the first and second commissure portions together during working cycles of the prosthetic valve.

2. The prosthetic valve of claim 1, wherein the retention member comprises a loop.

3. The prosthetic valve of claim 1, wherein the retention member comprises a suture.

4. The prosthetic valve of claim 1, wherein the retention member does not pierce any portions of the leaflets.

5. The prosthetic valve of claim 1, wherein the retention member extends continuously in a closed loop through the inner and outer fold layers of the first commissure portion, around radial inner edges of the inner fold layers of the first and second commissure portions, through the inner and outer fold layers of the second commissure portion and around radial outer edges of the inner fold layers of the first and second commissure portions.

6. The prosthetic valve of claim 1, wherein the retention member extends radially inwardly from the annular frame.

7. A prosthetic valve comprising:
an annular frame;
a plurality of leaflets positioned within and coupled to the annular frame, each leaflet comprising a free outflow edge and two opposing commissure portions on opposite sides of the leaflet, wherein each commissure portion is paired with a commissure portion of an adjacent leaflet to form a commissure that is coupled to the frame; and
for each commissure, a retention member coupled to two adjacent commissure portions to hold the adjacent commissure portions together during working cycles of the prosthetic valve, wherein the retention member extends in a radial inward direction relative to the annular frame.

8. The prosthetic valve of claim 7, wherein the retention member is a pre-formed, continuous band.

9. The prosthetic valve of claim 7, wherein the leaflets articulate at a location radially inward of the retention member.

10. The prosthetic valve of claim 7, wherein the retention member extends between an inner fold layer and an outer fold layer of a first commissure portion and between an inner fold layer and an outer fold layer of a second commissure portion to hold the first and second commissure portions together during a working cycle of the prosthetic valve.

11. The prosthetic valve of claim 10, wherein the retention member extends continuously in a closed loop through the inner and outer fold layers of the first commissure portion, around radial inner edges of the inner fold layers of the first and second commissure portions, through the inner and outer fold layers of the second commissure portion and around radial outer edges of the inner fold layers of the first and second commissure portions.

12. The prosthetic valve of claim 7, wherein the retention member is disposed in a horizontal plane that is perpendicular to a longitudinal axis of the annular frame.

13. The prosthetic valve of claim 7, wherein each commissure portion of each leaflet comprises two opposing primary tabs extending from opposite sides of the leaflet and two opposing secondary tabs disposed at opposite sides of the free outflow edge, wherein the retention member comprises stitches extending through a pair of adjacent secondary tabs of adjacent leaflets.

14. A method of assembling a prosthetic valve, the method comprising:
forming a valvular structure that includes a plurality of leaflets that define a plurality of commissures and a radially-extending retention member at each commissure; and
coupling the valvular structure to a frame.

15. The method of claim 14, wherein forming the valvular structure comprises:
for each leaflet of the plurality of leaflets, folding a commissure portion of the leaflet along a radially extending fold line to form an inner fold layer and an outer fold layer; and
positioning the retention member for each commissure between the inner fold layer and the outer fold layer of a first commissure portion of the commissure and between the inner fold layer and the outer fold layer of a second commissure portion of the commissure to hold the first and second commissure portions together during working cycles of the prosthetic valve.

16. A prosthetic valve comprising:
an annular frame;
a plurality of leaflets positioned within and coupled to the annular frame, each leaflet comprising a free outflow edge, two opposing primary tabs extending from opposite sides of the leaflet and two opposing secondary tabs disposed at opposite sides of the free outflow edge and extending axially beyond the free edge in downstream direction, wherein each primary tab is paired with a primary of an adjacent leaflet to form a commissure that is coupled to the frame; and
at each commissure, a retention member connecting a pair of adjacent secondary tabs of adjacent leaflets to hold the secondary tabs together during working cycles of the prosthetic valve.

17. The prosthetic valve of claim 16, wherein the leaflets articulate at a location radially inward of the retention member.

18. The prosthetic valve of claim 16, wherein the retention member comprises stitches that extend through a corresponding pair of secondary tabs.

19. The prosthetic valve of claim 16, wherein the retention member comprises an elongate member having two ends that are tied together to form a loop.

20. The prosthetic valve of claim 16, wherein the retention member is a pre-formed, continuous band.
